(19) United States
(12) Patent Application Publication
(10) Pub. No.: US 2020/0193858 A1
(43) Pub. Date: Jun. 18, 2020

(54) UNOBTRUSIVE MOTIVATION ESTIMATION

(71) Applicant: KONINKLIJKE PHILIPS N.V., Eindhoven (NL)

(72) Inventors: Murtaza BULUT, Eindhoven (NL); Saskia VAN DANTZIG, Utrecht (NL); Boris Emmanuel Rachmund DE RUYTER, Peer (BE); Anouk SCHROETEN, Eindhoven (NL); Jan Martijn KRANS, Deb Bosch (NL)

(21) Appl. No.: 16/710,751

(22) Filed: Dec. 11, 2019

Related U.S. Application Data
(60) Provisional application No. 62/778,488, filed on Dec. 12, 2018.

(51) Int. Cl. G09B 19/00; G16H 20/30; G09B 7/00

(52) U.S. Cl. CPC G09B 19/00; G09B 7/00; G16H 20/30

(57) ABSTRACT

In one embodiment, an apparatus, comprising: a memory comprising instructions; and one or more processors configured to execute the instructions to: receive first data corresponding to measurements of behavior of a user in the absence of a target behavior; receive second data corresponding to a measured behavior of the user relative to a target behavior, the target behavior representing an improvement in the behavior of the user corresponding to the first data; provide a first distribution for the second data; analyze the first distribution for asymmetry; estimate a level of motivation of the user based on the analysis; and provide the estimated level of motivation as a basis for a subsequent action.

### CROSS-REFERENCE TO PRIOR APPLICATIONS

This application claims the benefit of U.S. Provisional Application No. 62/778488, filed on 12 Dec. 2018.

### FIELD OF THE INVENTION

The present invention is generally related to digital coaching and engagement, and in particular, digital coaching and engagement using personal health applications.

### BACKGROUND OF THE INVENTION

Personal health applications use electronics devices, and typically portable electronics devices including wearable devices and/or smartphones, to provide for monitoring and/or rendering of consultation to users on a continual basis. For instance, a personal health application may deliver digital messages to the user via a phone or wearable interface that serves to inform of progress towards a goal and even influence behavior towards achieving that goal. Messages may be provided via personal apps running on the electronics device, or pushed from a remote server in communications with the electronics device. In either case, one objective is for the messages to actually be opened and reviewed by the user to enable the personal health application to help the user improve his or her health and/or well-being.

One illustration of a personal health application involves coaching and engagement applications, where an electronics device in possession of the user may monitor and/or receive data pertaining to physical activity and possible contextual information, and deliver digital messages to the user to assist the user in achieving a particular health goal based on the monitored progress, including losing weight, improving strength, and/or other health benefits.

People's development, performance and well-being are guided by their motivation. It is desirable to know the motivation type guiding people into action if their full potential is to be realized. Motivation is an especially relevant component for healthcare applications aiming at behavior change and engagement, such as medication adherence among chronic patients, long-term maintenance of weight loss among obese people, glucose control among diabetics, attendance and involvement in addiction-treatment programs, as well as lifestyle applications including involvement and maintaining sufficient physical activity and exercise, productivity, healthy eating, sleep, etc.

### SUMMARY OF THE INVENTION

In one embodiment, an apparatus, comprising: a memory comprising instructions; and one or more processors configured to execute the instructions to: receive first data corresponding to measurements of behavior of a user in the absence of a target behavior; receive second data corresponding to a measured behavior of the user relative to a target behavior, the target behavior representing an improvement in the behavior of the user corresponding to the first data; provide a first distribution for the second data; analyze the first distribution for asymmetry; estimate a level of motivation of the user based on the analysis; and provide the estimated level of motivation as a basis for a subsequent action.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that illustrates an example environment in which a motivation estimation system is used, in accordance with an embodiment of the invention.

FIG. 2 is a block diagram that illustrates an example wearable device that may implement all or a portion of the functionality of a motivation estimation system, in accordance with an embodiment of the invention.

FIG. 3 is a schematic diagram that illustrates an example electronics device that may implement all or a portion of the functionality of a motivation estimation system, in accordance with an embodiment of the invention.

FIG. 4 is a block diagram that illustrates an example computing device that may implement all or a portion of the functionality of a motivation estimation system, in accordance with an embodiment of the invention.

FIG. 5 is a block diagram that illustrates an example software architecture for implementing functionality of a motivation estimation system, in accordance with an embodiment of the invention.

FIG. 6 is a schematic diagram that illustrates an example distribution corresponding to an amotivated person, in accordance with an embodiment of the invention.

FIG. 7 is a schematic diagram that illustrates an example distribution corresponding to an extrinsically motivated person, in accordance with an embodiment of the invention.

FIG. 8 is a schematic diagram that illustrates an example distribution corresponding to an intrinsically motivated person, in accordance with an embodiment of the invention.

FIG. 9 is a flow diagram that illustrates an example of a motivation estimation method, in accordance with an embodiment of the invention.

### DETAILED DESCRIPTION OF EMBODIMENTS

Disclosed herein are certain embodiments of a motivation estimation system and method (collectively hereinafter referred to as a motivation estimation system) that unobtrusively quantify and measure a person's level and/or type of motivation. One rationale behind certain embodiments of a motivation estimation system is that it is possible to derive a person's level of motivation from the distribution of one or more behavioral parameters (e.g., daily step count) in relation to a behavioral target (e.g., to take 10,000 steps per day) and baseline behavior. An advantage over existing methods is that, at least for certain embodiments, there is no requirement for a user to fill out dedicated questionnaires (though in some circumstances, completion of questionnaires may improve function), and the system/method provides means for unobtrusive, continuous, and ubiquitous motivation assessment.

Digressing briefly, in some digital coaching and engagement programs, motivation is inferred from prior goal setting algorithms and/or past user behavior, and in particular, based on determining whether a selected goal setting algorithm has been successful or not. Such systems tend to consider motivation as a static user profile, where importance is attributed to the user's reaction to the goal setting method or perception of the goal. In contrast, certain embodiments of a motivation estimation system use motivation as a continuous parameter, computed continuously via a mathematical algorithm that uses distribution features pertaining to monitored behavior, enabling a more personalized coaching and engagement method and faster adaptation of the coaching and engagement methods according to a user's preferences.

Having summarized certain features of a motivation estimation system of the present disclosure, reference will now be made in detail to the description of a motivation estimation system as illustrated in the drawings. While a motivation estimation system will be described in connection with these drawings, there is no intent to limit it to the embodiment or embodiments disclosed herein. For instance, though emphasis is placed on a digital coaching and engagement program as an example health application (e.g., corresponding to physical activity, such as steps taken), it should be appreciated that some embodiments of a motivation estimation system may be used for other applications that intervene in human behavior. In other words, certain embodiments of a motivation estimation system may be applied to any behavior that people perform that is measurable and quantifiable and that is relative to a set target to be regularly or repeatedly met (e.g., on a daily basis), including physical activity (e.g., step count, active minutes, calorie expenditure, sedentary minutes, active breaks, etc.), food intake (e.g., calorie intake, glasses of water, etc.), productivity (e.g., work hours, minutes of distraction-free working, billable hours, minutes spent on social media, minutes slept with smartphone, minutes spent with other people, etc.), and sleep (e.g., total hours of sleep, bedtime, wake-up time, etc.). In some embodiments, in addition to, or in lieu of, using behaviors to estimate motivation, sensor data and/or signals corresponding to particular behaviors may be used to estimate motivation. For instance heart rate data may be used to define a target, including having a 50% higher heart rate than the mean heart rate for a given period (e.g., at last 10 minutes per day). In this example, a focus is not on a particular behavior, but rather, the data, as a user may engage in different activities or behaviors to achieve the target. As another illustrative example, an electroencephalogram (EEG) signal may be used, where a desire exists to achieve a certain percentage of alpha, delta, or theta waves per a period of time (e.g., per day), independent of the activity or behavior that the user engages in. Further, although the description identifies or describes specifics of one or more embodiments, such specifics are not necessarily part of every embodiment, nor are all of any various stated advantages necessarily associated with a single embodiment. On the contrary, the intent is to cover all alternatives, modifications and equivalents included within the principles and scope of the disclosure as defined by the appended claims. Further, it should be appreciated in the context of the present disclosure that the claims are not necessarily limited to the particular embodiments set out in the description.

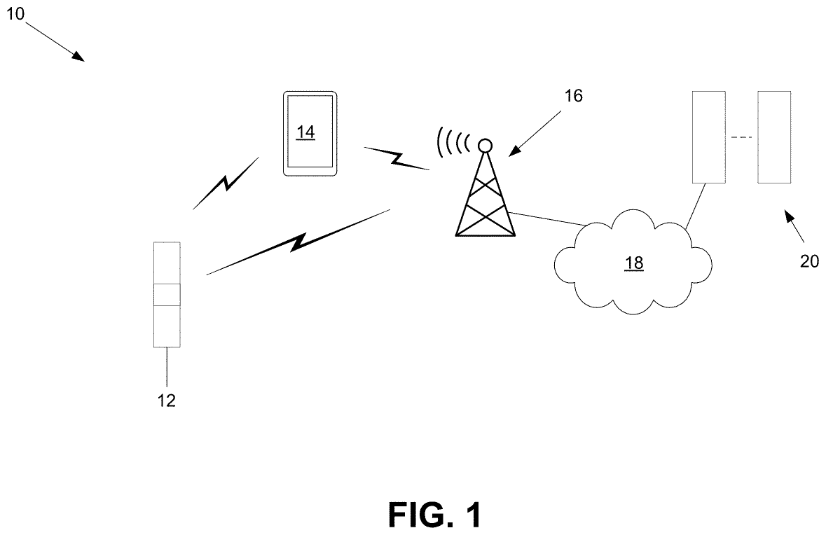

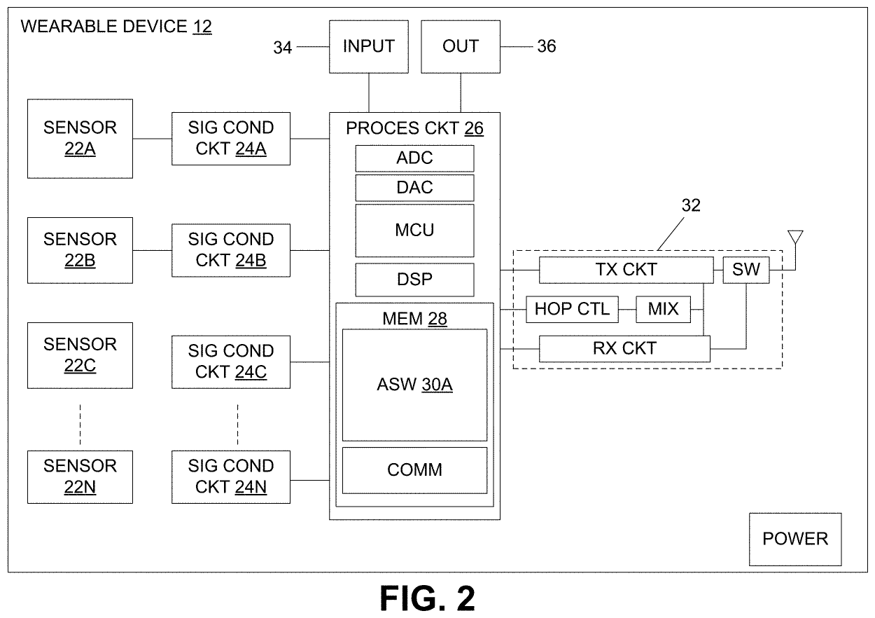

Referring now to FIG. 1, shown is an example environment 10 in which certain embodiments of a motivation estimation system may be implemented. It should be appreciated by one having ordinary skill in the art in the context of the present disclosure that the environment 10 is one example among many, and that some embodiments of a motivation estimation system may be used in environments with fewer, greater, and/or different components than those depicted in FIG. 1. The environment 10 comprises a plurality of devices that enable communication of information throughout one or more networks. The depicted environment 10 comprises a wearable device 12, one or more electronics devices 14 (one shown), a wireless/cellular network 16, a wide area network 18 (e.g., also described herein as the Internet, but also including, in some embodiments, one or more of an Internet of Things (IoT) network, an ambient intelligence network, among others), and a remote computing system 20. The wearable device 12, as described further in association with FIG. 2, and in one embodiment, is configured to perform all or at least a portion of the functionality of a motivation estimation system. The wearable device 12 is typically worn by the user (e.g., around the wrist, torso, or worn as a patch, or attached to an article of clothing), though some embodiments may embed the device 12 in the body (e.g., underneath the skin). In some embodiments, functionality of the wearable device 12 may be implemented in other types of devices, including house-based devices (e.g., channel control remotes, home smart-speaker assistants, etc.), autonomous vehicles/vehicle components (e.g., the steering wheel, stick, etc.), radios, alarm clocks, etc. In some embodiments, the wearable device 12 may include Google® glasses, wearable lenses, etc. using real time image capture, virtual, or augmented reality. The wearable device 12 comprises a plurality of sensors that track motion (e.g., steps, swim strokes, pedaling strokes, etc.) and/or physical activity (e.g., running, playing football, shopping, riding a bike, etc.) of the user, and sense/measure or derive physiological parameters (e.g., heart rate, respiration, skin temperature, etc.) based on the sensor data, and optionally sense various other parameters (e.g., contextual information, including outdoor temperature, humidity, location, etc.) pertaining to the surrounding environment of the wearable device 12. For instance, in some embodiments, the wearable device 12 may comprise a global navigation satellite system (GNSS) receiver, including a global positioning system (GPS) receiver, which tracks and provides location coordinates for the device 12. In some embodiments, the wearable device 12 may comprise indoor location technology, including beacons, RFID or other coded light technologies, wireless fidelity (Wi-Fi), etc., or other position tracking technology (e.g., using triangulation). Some embodiments of the wearable device 12 may include a motion or inertial tracking sensor, including an accelerometer and/or a gyroscope, providing movement data of the user. A representation of such gathered data may be communicated to the user via an integrated display on the wearable device 12 and/or on another device or devices.

Also, or alternatively, such data collected by the wearable device 12 may be communicated (e.g., continually, periodically, and/or aperiodically, including upon request) via a communications interface to one or more other devices, such as the electronics device 14 and/or (e.g., via the wireless/cellular network 16) to the computing system 20. Such communications may be achieved wirelessly (e.g., using near field communications (NFC) functionality, Blue-tooth functionality, 802.11-based technology, streaming technology, including LoRa, and/or broadband technology including 3G, 4G, 5G, etc.) and/or according to a wired medium (e.g., universal serial bus (USB), etc.). In some embodiments, the communications interface of the wearable device 12 may receive input from one or more devices, including the electronics device 14 and/or a device(s) of the computing system 20. Further discussion of the wearable device 12 is described below in association with FIG. 2.

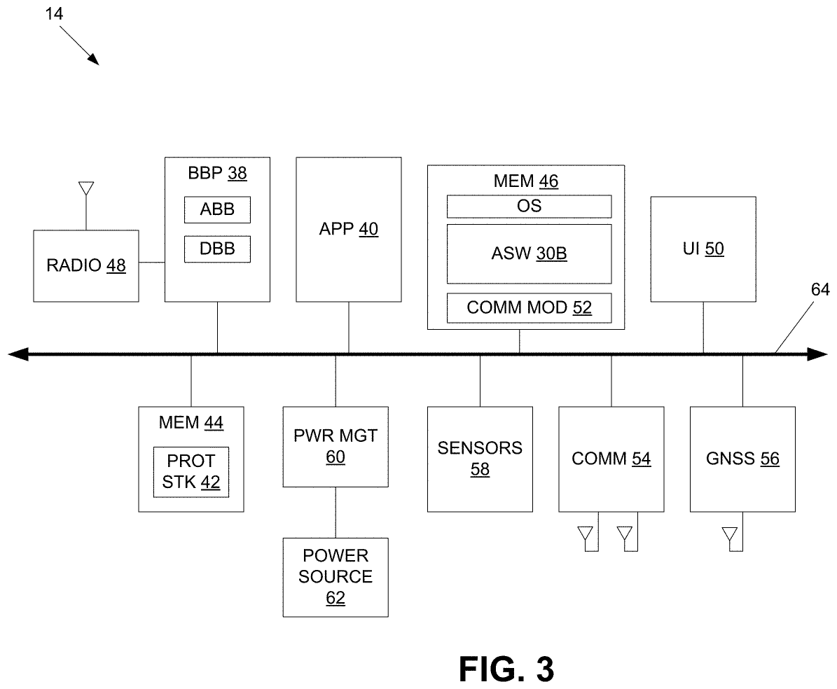

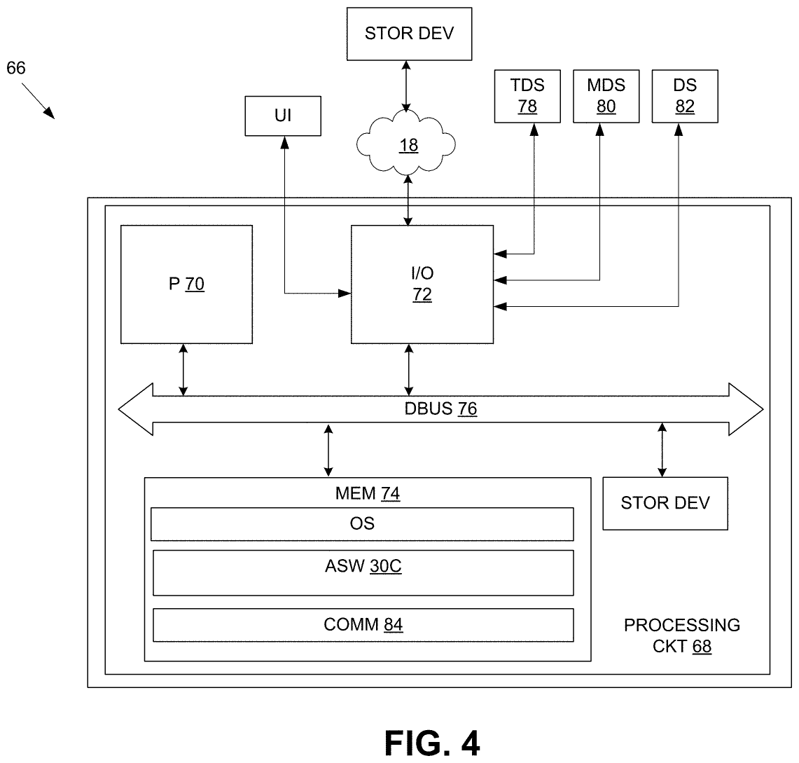

The electronics device 14 may be embodied as a smartphone, mobile phone, cellular phone, pager, stand-alone image capture device (e.g., camera), laptop, personal computer, workstation, among other handheld, portable, or other computing/communication devices, including communication devices having wireless communication capability, including telephony functionality. In the depicted embodiment of FIG. 1, the electronics device 14 is illustrated as a smartphone for convenience in illustration and description, though it should be appreciated that the electronics device 14 may take the form of other types of devices as explained above, including appliances (e.g., implementing the Internet of Things (IoT)), household devices, autonomous vehicles/vehicle components, etc. Further discussion of the electronics device 14 is described below in association with FIG. 3, with the terms smartphone and electronics device 14 used interchangeably hereinafter for convenience of illustration. Note that the architecture of a personal computer, laptop, workstation, etc. for the electronics device 14 may be similar to that described in connection with FIG. 4 in some embodiments. In one embodiment, the electronics device 14 is configured to perform all or at least a portion of a motivation estimation system. The electronics device 14 may be in communications with the wearable device 12 and/or one or more devices of the computing system 20, or the same or other types of electronics devices (e.g., smartphones, laptops, etc.). The electronics device 14 may include sensing functionality, including motion (e.g., acceleration) and/or physiological sensing. In one embodiment, the electronics device 14 comprises heart and/or breathing rate monitoring using a Philips Vital Signs Camera, or devices from other manufacturers with similar sensing functionality, to remotely measure heart and breathing rates using a standard, infrared (IR) based camera by sensing changes in skin color and body movement (e.g., chest movement), among others. The electronics device 14 may further include one or more interfaces for providing digital messaging, including a touch-type display screen. Wireless communication functionality, including cellular, streaming and/or broadband (e.g., 3G,4G, 5G, LoRa, etc.), Wi-Fi, Blue-tooth, NFC, etc., may be used for the communication of sensing data and/or digital messages among the devices 12, 14, and device(s) of the remote computing system 20), as explained further below in association with FIG. 3.

The wireless/cellular network 16 may include the necessary infrastructure to enable wireless and/or cellular communications between the wearable device 12, the electronics device 14, and one or more devices of the remote computing system 20. There are a number of different digital cellular technologies suitable for use in the wireless/cellular network 16, including: 3G, 4G, 5G, GSM, GPRS, CDMAOne, CDMA2000, Evolution-Data Optimized (EV-DO), EDGE, Universal Mobile Telecommunications System (UMTS), Digital Enhanced Cordless Telecommunications (DECT), Digital AMPS (IS-136/TDMA), and Integrated Digital Enhanced Network (iDEN), among others, as well as Wireless-Fidelity (W-Fi), 802.11, streaming, etc., for some example wireless technologies,

The wide area network 18 may comprise one or a plurality of networks that in whole or in part comprise the Internet. The wearable device 12 and/or the electronics device 14 may access one or more of the devices of the computing system 20 via the wireless/cellular network 16 and/or the Internet 18, which may be further enabled through access to one or more networks including PSTN (Public Switched Telephone Networks), POTS, Integrated Services Digital Network (ISDN), Ethernet, Fiber, DSL/ADSL, Wi-Fi, among others. For wireless implementations, the wireless/cellular network 16 may use wireless fidelity (Wi-Fi) to receive data converted by the wearable device 12 and/or the electronics device 14 to a radio format and process (e.g., format) for communication over the Internet 18. The wireless/cellular network 16 may comprise suitable equipment that includes a modem, router, etc.

The computing system 20 comprises one or more devices coupled to the wide area network 18, including one or more computing devices networked together, including an application server(s) and data storage. The computing system 20 may serve as a cloud computing environment (or other server network) for the wearable device 12 and/or the electronics device 14, performing processing and/or data storage on behalf of (or in some embodiments, in addition to) the wearable device 12 and/or the electronics device 14. One or more devices of the computing system 20 may implement all or at least a portion of certain embodiments of a motivation estimation system. In one embodiment, the computing system 20 may be configured to be a backend server for a health engagement program. The computing system 20 receives observations (e.g., data) collected via sensors or input interfaces of one or more of the wearable device 12 or electronics device 14 and/or other devices or applications (e.g., third party internet services that provide, for instance, weather reports/forecasts to enable intelligent decisions on whether to recommend an outdoor activity, location services (e.g., Google maps) that provide geospatial data to be used in combination with the received location information (e.g., GPS data) for ascertaining environmental information (e.g., presence of sidewalks), stores the received data in a data structure (e.g., user profile database, etc.), and generates digital messages, including notifications or signals to activate or cause the activation of haptic, light-emitting, and/or aural-based devices or hardware components, among other actions) for presentation to the user. The computing system 20 is programmed to handle the operations of one or more health or wellness engagement programs implemented on the wearable device 12 and/or electronics device 14 via the networks 16 and/or 18. For example, the computing system 20 processes user registration requests, user device activation requests, user information updating requests, data uploading requests, data synchronization requests, etc. The data received at the computing system 20 may be a plurality of measurements pertaining to the parameters, for example, body movements and activities/behavior, heart rate, respiration rate, blood pressure, body temperature, light and visual information, etc., user feedback/input, and the corresponding context. Based on the data observed during a period of time and/or over a large population of users, the computing system 20 may generate messages pertaining to each specific parameter or a combination of parameters, and provide the messages via the networks 16 and/or 18 for presentation on devices 12 and/or 14. In some embodiments, the computing system 20 is configured to be a backend server for a health-related program or a health-related application implemented on the mobile devices. The functions of the computing system 20 described above are for illustrative purpose only. The present disclosure is not intended to be limiting. The computing system 20 may be a general computing server or a dedicated computing server. The computing system 20 may be configured to provide backend support for a program developed by a specific manufacturer.

When embodied as a cloud service or services, the device(s) of the remote computing system 20 may comprise an internal cloud, an external cloud, a private cloud, a public cloud (e.g., commercial cloud), or a hybrid cloud, which includes both on-premises and public cloud resources. For instance, a private cloud may be implemented using a variety of cloud systems including, for example, Eucalyptus Systems, VMWare vSphere®, or Microsoft® HyperV. A public cloud may include, for example, Amazon EC2®, Amazon Web Services®, Terremark®, Savvis®, or GoGrid®. Cloud-computing resources provided by these clouds may include, for example, storage resources (e.g., Storage Area Network (SAN), Network File System (NFS), and Amazon S3®), network resources (e.g., firewall, load-balancer, and proxy server), internal private resources, external private resources, secure public resources, infrastructure-as-a-services (IaaSs), platform-as-a-services (PaaSs), or software-as-a-services (SaaSs). The cloud architecture of the devices of the remote computing system 20 may be embodied according to one of a plurality of different configurations. For instance, if configured according to MICROSOFT AZURE™, roles are provided, which are discrete scalable components built with managed code. Worker roles are for generalized development, and may perform background processing for a web role. Web roles provide a web server and listen for and respond to web requests via an HTTP (hypertext transfer protocol) or HTTPS (HTTP secure) endpoint. VM roles are instantiated according to tenant defined configurations (e.g., resources, guest operating system). Operating system and VM updates are managed by the cloud. A web role and a worker role run in a VM role, which is a virtual machine under the control of the tenant. Storage and SQL services are available to be used by the roles. As with other clouds, the hardware and software environment or platform, including scaling, load balancing, etc., are handled by the cloud.

In some embodiments, the devices of the remote computing system 20 may be configured into multiple, logically-grouped servers (run on server devices), referred to as a server farm. The devices of the remote computing system 20 may be geographically dispersed, administered as a single entity, or distributed among a plurality of server farms, executing one or more applications on behalf of, or processing data from, one or more of the wearable device 12 and/or the electronics device 14. The devices of the remote computing system 20 within each farm may be heterogeneous. One or more of the devices may operate according to one type of operating system platform (e.g., WINDOWS NT, manufactured by Microsoft Corp. of Redmond, Wash.), while one or more of the other devices may operate according to another type of operating system platform (e.g., Unix or Linux). The group of devices of the remote computing system 20 may be logically grouped as a farm that may be interconnected using a wide-area network (WAN) connection or medium-area network (MAN) connection. The devices of the remote computing system 20 may each be referred to as, and operate according to, a file server device, application server device, web server device, proxy server device, or gateway server device.

In one embodiment, the computing system 20 may comprise a web server that provides a web site that can be used by users to review information related to monitored behavior/activity and/or review/update user data and/or a record of measurements. The computing system 20 may receive data collected via one or more of the wearable device 12 and/or the electronics device 14, store the received data in a data structure (e.g., user profile database) along with one or more tags, process the information (e.g., to determine a behavior and appropriate action, including an appropriate message), and deliver the message at one or more of the devices 12 and/or 14 and/or take or cause other and/or additional actions. The computing system 20 is programmed to handle the operations of one or more health or wellness engagement programs implemented on the wearable device 12 and/or electronics device 14 via the networks 16 and/or 18. For example, the computing system 20 processes user registration requests, user device activation requests, user information updating requests, data uploading requests, data synchronization requests, etc. In one embodiment, the data received at the computing system 20 may be stored in a user profile data structure comprising a plurality of measurements pertaining to activity/inactivity, for example, body movements, sensed physiological measurements, including heart rate (e.g., average heart rate, heart rate variations), heart rhythm, inter-beat interval, respiration rate, blood pressure, body temperature, etc., context (e.g., location, environmental conditions, etc. tagged to one or more of the measurements), and/or a history of feedback messages. In some embodiments, the computing system 20 is configured to serve as a backend server for a health-related engagement program or a health-related engagement application implemented on the wearable device 12 and/or the electronics device 14. The functions of the computing system 20 described above are for illustrative purpose only. The present disclosure is not intended to be limiting. The computing system 20 may be a general computing server device or a dedicated computing server device. The computing system 20 may be configured to provide backend support for a program developed by a specific manufacturer. However, the computing system 20 may also be configured to be interoperable across other server devices and generate information in a format that is compatible with other programs. In some embodiments, one or more of the functionality of the computing system 20 may be performed at the respective devices 12 and/or 14.

Note that cooperation between the wearable device 12 and/or the electronics device 14 and the one or more devices of the computing system 20 may be facilitated (or enabled) through the use of one or more application programming interfaces (APIs) that may define one or more parameters that are passed between a calling application and other software code such as an operating system, a library routine, and/or a function that provides a service, that provides data, or that performs an operation or a computation. The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer employs to access functions supporting the API. In some implementations, an API call may report to an application the capabilities of a device running the application, including input capability, output capability, processing capability, power capability, and communications capability. Further discussion of an example device of the computing system 20 is described below in association with FIG. 4.

An embodiment of a motivation estimation system may comprise the wearable device 12, the electronics device 14, and/or the computing system 20. In other words, one or more of the aforementioned devices 12, 14, and device(s) of the remote computing system 20 may implement the functionality of a motivation estimation system. For instance, the wearable device 12 may comprise all of the functionality of a motivation estimation system, enabling the user to avoid or limit the need for Internet connectivity and/or any inconvenience in carrying a smartphone 14 around. In some embodiments, the functionality of a motivation estimation system may be implemented using any combination of the wearable device 12, the electronics device 14, and/or the computing system 20. For instance, the wearable device 12 may provide for sensing functionality and a rudimentary feedback capability, the electronics device 14 may provide a more sophisticated interface for the presentation of messages, monitoring functionality for when messages are opened and/or read by the user, and serve as a communications intermediary between the computing system 20 and the wearable device 12, and the computing system 20 may receive (e.g., from the wearable device 12 and/or the smartphone 14) the measurement and/or contextual data (and possibly indications of when a user opens messages) from the devices 12, 14 and responsively provide messages (e.g., coaching messages) to the electronics device 14 for presentation. These and/or other variations, including distributed processing, measurement, etc., may be used among the devices/system 12, 14, and/or 20, and hence are contemplated to be within the scope of the disclosure.

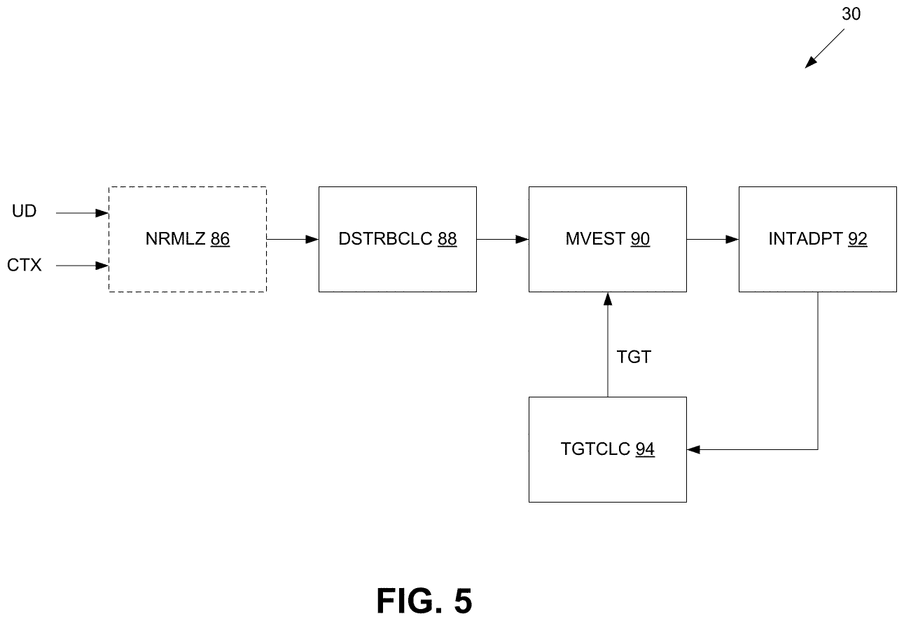

Having generally described an example environment 10 in which an embodiment of a motivation estimation system may be implemented, attention is directed to FIG. 2. FIG. 2 illustrates example circuitry for the example wearable device 12, and in particular, underlying circuitry and software (e.g., architecture) of the wearable device 12 that in one embodiment is used to implement a motivation estimation system. It should be appreciated by one having ordinary skill in the art in the context of the present disclosure that the architecture of the wearable device 12 depicted in FIG. 2 is but one example, and that in some embodiments, additional, fewer, and/or different components may be used to achieve similar and/or additional functionality. In one embodiment, the wearable device 12 comprises a plurality of sensors 22 (e.g., 22A-22N), one or more signal conditioning circuits 24 (e.g., SIG COND CKT 24A-SIG COND CKT 24N) coupled respectively to the sensors 22, and a processing circuit 26 (PROCES CKT) that receives the conditioned signals from the signal conditioning circuits 24. In one embodiment, the processing circuit 26 comprises an analog-to-digital converter (ADC), a digital-to-analog converter (DAC), a microcontroller unit (MCU), a digital signal processor (DSP), and memory (MEM) 28. In some embodiments, the processing circuit 26 may comprise fewer or additional components than those depicted in FIG. 2. For instance, in one embodiment, the processing circuit 26 may consist exclusively or primarily of the microcontroller. In some embodiments, the processing circuit 26 may include the signal conditioning circuits 24. The memory 28 comprises an operating system (OS) and application software (ASW) 30A. The application software 30A comprises instructions/executable code to implement all or a portion of the motivation estimation system, as is described further below in conjunction with FIG. 5. The memory 28 also comprises communications software (COMM), such as that used to enable the wearable device 12 to operate according to one or more of a plurality of different communication technologies (e.g., GSM, WCDMA, 3G, 4G,5G, streaming (e.g., LoRa), NFC, Bluetooth, Wi-Fi, Zigbee, etc.). In some embodiments, the communications software may be part of the application software 30A or located in separate or other memory.

In one embodiment, the processing circuit 26 is coupled to a communications circuit 32. The communications circuit 32 serves to enable wireless communications between the wearable device 12 and other devices, including the electronics device 14 and the computing system 20, among other devices. The communications circuit 32 is depicted as a Bluetooth circuit, though not limited to this transceiver configuration. For instance, in some embodiments, the communications circuit 32 may be embodied as one or any combination of an NFC circuit, Wi-Fi circuit, transceiver circuitry based on Zigbee, 802.11, GSM, LTE, CDMA, WCDMA, circuitry for enabling broadband and/or streaming (e.g., 3G, 4G, 5G, LoRa, etc.), among others including optical or ultrasonic based technologies. The processing circuit 26 is further coupled to input/output (I/O) devices or peripherals, including an input interface 34 (INPUT) and an output interface 36 (OUT). Note that in some embodiments, functionality for one or more of the aforementioned circuits and/or software may be combined into fewer components/modules, or in some embodiments, further distributed among additional components/modules or devices. For instance, the processing circuit 26 may be packaged as an integrated circuit that includes the microcontroller (microcontroller unit or MCU), the DSP, and memory 28, whereas the ADC and DAC may be packaged as a separate integrated circuit coupled to the processing circuit 26. In some embodiments, one or more of the functionality for the above-listed components may be combined, such as functionality of the DSP performed by the microcontroller.

The sensors 22 perform detection and measurement of a plurality of physiological and behavioral parameters (e.g., typical behavioral parameters or activities including walking, running, cycling, and/or other activities, including shopping, walking a dog, working in the garden, taking medicine, watching TV, etc.), including heart rate, heart rate variability, heart rate recovery, blood flow rate, activity level, muscle activity (e.g., movement of limbs, repetitive movement, core movement, body orientation/position, power, speed, acceleration, etc.), muscle tension, blood volume, blood pressure, blood oxygen saturation, respiratory rate, perspiration, skin temperature, brain waves (e.g., for sleep tracking/monitoring and/or relaxation/meditation tracking, etc.), body weight, and body composition (e.g., body fat percentage, etc.). At least one of the sensors 22 may be embodied as a movement detecting sensor, including an inertial sensor (e.g., gyroscope, single or multi-axis accelerometer, such as one using piezoelectric, piezoresistive or capacitive technology in a microelectromechanical system (MEMS) infrastructure for sensing movement) and/or as GNSS sensor, including a GPS receiver to facilitate determinations of distance, speed, acceleration, location, altitude, etc. (e.g., location data, or generally, sensing movement), in addition to or in lieu of the accelerometer/gyroscope and/or indoor tracking (e.g., WiFi, coded-light based technology, acoustic-based tracking, etc.) and/or other tracking/location mechanisms. The sensors 22 may also include flex and/or force sensors (e.g., using variable resistance), electromyographic sensors, electrocardiographic sensors (e.g., EKG, ECG) magnetic sensors, photoplethysmographic (PPG) sensors, bio-impedance sensors, infrared proximity sensors, acoustic/ultrasonic/audio sensors, a strain gauge, galvanic skin/sweat sensors, pH sensors, temperature sensors, pressure sensors, electroencephalography sensors (EEG) and photocells. The sensors 22 may include other and/or additional types of sensors for the detection of, for instance, environmental conditions including barometric pressure, humidity, outdoor temperature, etc. In some embodiments, GNSS functionality may be achieved via the communications circuit 32 or other circuits coupled to the processing circuit 26.

The signal conditioning circuits 24 include amplifiers and filters, among other signal conditioning components, to condition the sensed signals including data corresponding to the sensed physiological parameters and/or location signals before further processing is implemented at the processing circuit 26. Though depicted in FIG. 2 as respectively associated with each sensor 22, in some embodiments, fewer signal conditioning circuits 24 may be used (e.g., shared for more than one sensor 22) or fewer sensors 22 may be used. In some embodiments, the signal conditioning circuits 24 (or functionality thereof) may be incorporated elsewhere, such as in the circuitry of the respective sensors 22 or in the processing circuit 26 (or in components residing therein). Further, although described above as involving unidirectional signal flow (e.g., from the sensor 22 to the signal conditioning circuit 24), in some embodiments, signal flow may be bi-directional. For instance, in the case of optical measurements, the microcontroller may cause an optical signal to be emitted from a light source (e.g., light emitting diode(s) or LED(s)) in or coupled to the circuitry of the sensor 22, with the sensor 22 (e.g., photocell) receiving the reflected/refracted signals.

The communications circuit 32 is managed and controlled by the processing circuit 26 (e.g., executing the communications software), though in some embodiments, the communications circuit 32 may be implemented without software control. The communications circuit 32 is used to wirelessly interface with the electronics device 14 (FIG. 3) and/or one or more devices of the computing system 20 (FIG. 4). In one embodiment, the communications circuit 32 may be configured as a Bluetooth (including BTLE) transceiver, though in some embodiments, other and/or additional technologies may be used, such as Wi-Fi, 3G, 4G, 5G, GSM, LTE, CDMA and its derivatives, Zigbee, NFC, streaming, among others. In the embodiment depicted in FIG. 2, the communications circuit 32 comprises a transmitter circuit (TX CKT), a switch (SW), an antenna, a receiver circuit (RX CKT), a mixing circuit (MIX), and a frequency hopping controller (HOP CTL). The transmitter circuit and the receiver circuit comprise components suitable for providing respective transmission and reception of an RF signal, including a modulator/demodulator, filters, and amplifiers. In some embodiments, demodulation/modulation and/or filtering may be performed in part or in whole by the DSP. The switch switches between receiving and transmitting modes. The mixing circuit may be embodied as a frequency synthesizer and frequency mixers, as controlled by the processing circuit 26. The frequency hopping controller controls the hopping frequency of a transmitted signal based on feedback from a modulator of the transmitter circuit. In some embodiments, functionality for the frequency hopping controller may be implemented by the microcontroller or DSP. Control for the communications circuit 32 may be implemented by the microcontroller, the DSP, or a combination of both. In some embodiments, the communications circuit 32 may have its own dedicated controller that is supervised and/or managed by the microcontroller.

In one example operation, a signal (e.g., at 2.4 GHz) may be received at the antenna and directed by the switch to the receiver circuit. The receiver circuit, in cooperation with the mixing circuit, converts the received signal into an intermediate frequency (IF) signal under frequency hopping control attributed by the frequency hopping controller and then to baseband for further processing by the ADC. On the transmitting side, the baseband signal (e.g., from the DAC of the processing circuit 26) is converted to an IF signal and then RF by the transmitter circuit operating in cooperation with the mixing circuit, with the RF signal passed through the switch and emitted from the antenna under frequency hopping control provided by the frequency hopping controller. The modulator and demodulator of the transmitter and receiver circuits may be frequency shift keying (FSK) type modulation/demodulation, though not limited to this type of modulation/demodulation, which enables the conversion between IF and baseband. In some embodiments, demodulation/modulation and/or filtering may be performed in part or in whole by the DSP. The memory 28 stores the communications software, which when executed by the microcontroller, controls the Bluetooth (and/or other protocols) transmission/reception.

The communications circuit 32 may be implemented as an IF-type transceiver, or in some embodiments, a direct conversion architecture may be implemented. As noted above, the communications circuit 32 may be embodied according to other and/or additional transceiver technologies.

The processing circuit 26 is depicted in FIG. 2 as including the ADC and DAC. For sensing functionality, the ADC converts the conditioned signal from the signal conditioning circuit 24 and digitizes the signal for further processing by the microcontroller and/or DSP. The ADC may also be used to convert analogs inputs that are received via the input interface 34 to a digital format for further processing by the microcontroller. The ADC may also be used in baseband processing of signals received via the communications circuit 32. The DAC converts digital information to analog information. Its role for sensing functionality may be to control the emission of signals, such as optical signals or acoustic signal, from the sensors 22. The DAC may further be used to cause the output of analog signals from the output interface 36. Also, the DAC may be used to convert the digital information and/or instructions from the microcontroller and/or DSP to analog signals that are fed to the transmitter circuit. In some embodiments, additional conversion circuits may be used.

The microcontroller and the DSP provide the processing functionality for the wearable device 12. In some embodiments, functionality of both processors may be combined into a single processor, or further distributed among additional processors. The DSP provides for specialized digital signal processing, and enables an offloading of processing load from the microcontroller. The DSP may be embodied in specialized integrated circuit(s) or as field programmable gate arrays (FPGAs). In one embodiment, the DSP comprises a pipelined architecture, which comprises a central processing unit (CPU), plural circular buffers and separate program and data memories according to, say, a Harvard architecture. The DSP further comprises dual busses, enabling concurrent instruction and data fetches. The DSP may also comprise an instruction cache and I/O controller, such as those found in Analog Devices SHARC® DSPs, though other manufacturers of DSPs may be used (e.g., Freescale multi-core MSC81xx family, Texas Instruments C6000 series, etc.). The DSP is generally utilized for math manipulations using registers and math components that may include a multiplier, arithmetic logic unit (ALU, which performs addition, subtraction, absolute value, logical operations, conversion between fixed and floating point units, etc.), and a barrel shifter. The ability of the DSP to implement fast multiply-accumulates (MACs) enables efficient execution of Fast Fourier Transforms (FFTs) and Finite Impulse Response (FIR) filtering. Some or all of the DSP functions may be performed by the microcontroller. The DSP generally serves an encoding and decoding function in the wearable device 12. For instance, encoding functionality may involve encoding commands or data corresponding to transfer of information to the electronics device 14 or a device of the computing system 20. Also, decoding functionality may involve decoding the information received from the sensors 22 (e.g., after processing by the ADC).

The microcontroller comprises a hardware device for executing software/firmware, particularly that stored in memory 28. The microcontroller can be any custom made or commercially available processor, a central processing unit (CPU), a semiconductor based microprocessor (in the form of a microchip or chip set), a macroprocessor, or generally any device for executing software instructions. Examples of suitable commercially available microprocessors include Intel's® Itanium® and Atom® microprocessors, to name a few non-limiting examples. The microcontroller provides for management and control of the wearable device 12, including determining physiological parameters and/or location coordinates or other contextual information based on the sensors 22, and for enabling communication with the electronics device 14 and/or a device of the computing system 20, and in some embodiments, for the presentation of messages and/or implementation or triggering of other actions.

The memory 28 can include any one or combination of volatile memory elements (e.g., random access memory (RAM, such as DRAM, SRAM, SDRAM, etc.)) and nonvolatile memory elements (e.g., ROM, Flash, solid state, EPROM, EEPROM, etc.). Moreover, the memory 28 may incorporate electronic, magnetic, and/or other types of storage media.

The software in memory 28 may include one or more separate programs, each of which comprises an ordered listing of executable instructions for implementing logical functions. In the example of FIG. 2, the software in the memory 28 includes a suitable operating system and the application software 30A, which may run (or work in conjunction with) a health engagement program and further includes one or more algorithms for determining physiological and/or behavioral measures and/or other information (e.g., including location, speed of travel, environmental, etc.) based on the output from the sensors 22, and motivational levels based on data distribution generation and analysis. The raw data from the sensors 22 may be used by the algorithms to determine various physiological and/or behavioral measures (e.g., heart rate, biomechanics, such as swinging of the arms, steps taken, etc.), and may also be used to derive other parameters, such as energy expenditure, heart rate recovery, aerobic capacity (e.g., VO2 max, etc.), among other derived measures of physical performance. In some embodiments, these derived parameters may be computed externally (e.g., at the electronics devices 14 or one or more devices of the computing system 20) in lieu of, or in addition to, the computations performed local to the wearable device 12. In some embodiments, the GPS functionality of the sensors 22 collects contextual data (time and location data, including location coordinates, weather, etc.). The application software 30A may also collect information about the means of ambulation. For instance, the GPS data (which may include time coordinates) may be used by the application software 30A to determine speed of travel, which may indicate whether the user is moving within a vehicle, on a bicycle, or walking or running. In some embodiments, other and/or additional data may be used to assess the type of activity, including physiological data (e.g., heart rate, respiration rate, galvanic skin response, etc.) and/or behavioral data. In some embodiments, the application software 30A further comprises software to provide messages (e.g., generated at the wearable device 12 or at another device(s), including the electronics device 14 and/or a device of the computing system 20). In some embodiments, the application software 30A performs, or triggers the implementation of, other and/or additional functions, including adapting one or more intervention parameters (e.g., target behavior, messaging characteristics, communication characteristics, coaching characteristics, etc.) and/or replacement of software modules (e.g., replacing a running intervention module with a module pertaining to a different function).

The operating system essentially controls the execution of other computer programs, such as the application software 30A, and provides scheduling, input-output control, file and data management, memory management, and communication control and related services. The memory 28 may also include user data, including weight, height, age, gender, goals, body mass index (BMI) that are used by the microcontroller executing the executable code of the algorithms to accurately interpret the measured physiological and/or behavioral data. The user data may also include historical data relating past recorded data to prior contexts (e.g., environmental conditions, user state, etc.), and/or in some embodiments, past messages and/or message characteristics (e.g., including type of message, format, frequency of delivery, message distribution, delivery channel(s), times of delivery, associated cards used for the delivery mechanism and respective features, use of the message (e.g., whether links were selected, read, etc.), past responses to messages, etc.). In some embodiments, the user data may include responses to questionnaires that are fashioned to learn the motivation level of the user, as described below. In some embodiments, the memory 28 may store other data, including information about the status of the network, the periods the network used for communication was down or working properly, signal strength, among other parameters related to the medium of communication and/or the signals. In some embodiments, one or more of the historical data and/or other information may be stored at one or more other devices. In some embodiments, the application software 30A may comprise learning algorithms, data mining functionality, among other features.

Although the application software 30A is described above as being implemented in the wearable device 12, some embodiments may distribute the corresponding functionality among the wearable device 12 and other devices (e.g., electronics device 14 and/or one or more devices of the computing system 20 and/or a vehicle), or in some embodiments, the application software 30A may be implemented in another device (e.g., the electronics device 14, a device of the computing system 20).

The software in memory 28 comprises a source program, executable program (object code), script, or any other entity comprising a set of instructions to be performed. When a source program, then the program may be translated via a compiler, assembler, interpreter, or the like, so as to operate properly in connection with the operating system. Furthermore, the software can be written as (a) an object oriented programming language, which has classes of data and methods, or (b) a procedure programming language, which has routines, subroutines, and/or functions, for example but not limited to, C, C++, Python, Java, among others. The software may be embodied in a computer program product, which may be a non-transitory computer readable medium or other medium.

The input interface 34 comprises an interface (e.g., including a user interface) for entry of user input, such as a button or microphone or sensor (e.g., to detect user input, including gestures, physiological signals, etc.) or touch-type display. In some embodiments, the input interface 34 may comprise a communications port for downloaded information to the wearable device 12 (such as via a wired connection). The output interfaces 36 comprises an interface for the presentation or transfer of data, including a user interface (e.g., display screen presenting a graphical user interface) or communications interface for the transfer (e.g., wired) of information stored in the memory, or to enable one or more feedback devices, such as lighting devices (e.g., LEDs), audio devices (e.g., tone generator and speaker), and/or tactile feedback devices (e.g., vibratory motor). For instance, the output interface 36 may be used to present messages to the user. In some embodiments, at least some of the functionality of the input and output interfaces 34 and 36, respectively, may be combined, including being embodied at least in part as a touch-type display screen for the entry of input (including replies to questionnaires) and to provide digital messages (e.g., coaching messages, questionnaires, etc.). The wearable device 12 may also include a power source (POWER), such as a battery.

Referring now to FIG. 3, shown is an example electronics device 14 in which all or a portion of the functionality of a motivation estimation system may be implemented. In the depicted example, the electronics device 14 is embodied as a smartphone (hereinafter, referred to as smartphone 14 for illustration and convenience), though in some embodiments, other types of devices may be used, such as a workstation, laptop, notebook, tablet, home appliance, vehicle/vehicle component, etc. It should be appreciated by one having ordinary skill in the art that the logical block diagram depicted in FIG. 3 and described below is one example, and that other designs may be used in some embodiments. The smartphone 14 comprises application software (ASW) 30B, which may include all or a portion of the functionality of a motivation estimation system. Functionality of certain embodiments of a motivation estimation system may be carried out entirely using the smartphone 14, or in some embodiments, carried out in part with the cooperation of additional devices of the environment 10 (FIG. 1). The smartphone 14 comprises at least two different processors, including a baseband processor (BBP) 38 and an application processor (APP) 40. As is known, the baseband processor 38 primarily handles baseband communication-related tasks and the application processor 40 generally handles inputs and outputs and all applications other than those directly related to baseband processing. The baseband processor 38 comprises a dedicated processor for deploying functionality associated with a protocol stack (PROT STK) 42, such as a GSM (Global System for Mobile communications) protocol stack, among other functions. The application processor 40 comprises a multi-core processor for running applications, including all or a portion of the application software 30B. The baseband processor 38 and application processor 40 have respective associated memory (e.g., MEM) 44, 46, including random access memory (RAM), Flash memory, etc., and peripherals, and a running clock. Note that, though depicted as residing in memory 46, all or a portion of the application software 30B may be stored in memory 44, distributed among memory 44, 46, or reside in other memory.

More particularly, the baseband processor 38 may deploy functionality of the protocol stack 42 to enable the smartphone 14 to access one or a plurality of wireless network technologies, including WCDMA (Wideband Code Division Multiple Access), CDMA (Code Division Multiple Access), EDGE (Enhanced Data Rates for GSM Evolution), broadband (e.g., 3G,4G,5G), streaming services (e.g., LoRa), GPRS (General Packet Radio Service), Zigbee (e.g., based on IEEE 802.15.4), Bluetooth, Wi-Fi (Wireless Fidelity, such as based on IEEE 802.11), and/or LTE (Long Term Evolution), among variations thereof and/or other telecommunication protocols, standards, and/or specifications.

The baseband processor 38 manages radio communications and control functions, including signal modulation, radio frequency shifting, and encoding. The baseband processor 38 comprises, or may be coupled to, a radio (e.g., RF front end) 48 and/or a GSM modem, and analog and digital baseband circuitry (ABB, DBB, respectively in FIG. 3). The radio 48 comprises one or more antennas, a transceiver, and a power amplifier to enable the receiving and transmitting of signals of a plurality of different frequencies, enabling access to the wireless/cellular network 16 (FIG. 1), and hence sending or receiving communications involving user data, measurements, associated contexts, and/or messages. The analog baseband circuitry is coupled to the radio 48 and provides an interface between the analog and digital domains of the GSM modem (and/or cellular modem of other standards/specifications/protocols). The analog baseband circuitry comprises circuitry including an analog-to-digital converter (ADC) and digital-to-analog converter (DAC), as well as control and power management/distribution components and an audio codec to process analog and/or digital signals received indirectly via the application processor 40 or directly from the smartphone user interface (UI) 50 (e.g., microphone, speaker, earpiece, ring tone, vibrator circuits, display screen, etc.). The ADC digitizes any analog signals for processing by the digital baseband circuitry. The digital baseband circuitry deploys the functionality of one or more levels of the GSM protocol stack (e.g., Layer 1, Layer 2, etc.), and comprises a microcontroller (e.g., microcontroller unit or MCU, also referred to herein as a processor) and a digital signal processor (DSP, also referred to herein as a processor) that communicate over a shared memory interface (the memory comprising data and control information and parameters that instruct the actions to be taken on the data processed by the application processor 40). The MCU may be embodied as a RISC (reduced instruction set computer) machine that runs a real-time operating system (RTIOS), with cores having a plurality of peripherals (e.g., circuitry packaged as integrated circuits) such as RTC (real-time clock), SPI (serial peripheral interface), I2C (inter-integrated circuit), UARTs (Universal Asynchronous Receiver/Transmitter), devices based on IrDA (Infrared Data Association), SD/MMC (Secure Digital/Multimedia Cards) card controller, keypad scan controller, and USB devices, GPRS crypto module, TDMA (Time Division Multiple Access), smart card reader interface (e.g., for the one or more SIM (Subscriber Identity Module) cards), timers, and among others. For receive-side functionality, the MCU instructs the DSP to receive, for instance, in-phase/quadrature (I/Q) samples from the analog baseband circuitry and perform detection, demodulation, and decoding with reporting back to the MCU. For transmit-side functionality, the MCU presents transmittable data and auxiliary information to the DSP, which encodes the data and provides the encoded data to the analog baseband circuitry (e.g., converted to analog signals by the DAC).

The application processor 40 operates under control of an operating system (OS) that enables the implementation of one or a plurality of user applications, including the application software 30B and a health or coaching and engagement application. The application processor 40 may be embodied as a System on a Chip (SOC), and supports a plurality of multimedia related features including web browsing functionality to access one or more computing devices of the computing system 20 (FIG. 1) that are coupled to the Internet, email, multimedia entertainment, games, etc. For instance, the application processor 40 may execute communications module 52, which may include middleware (e.g., browser with or operable in association with one or more application program interfaces (APIs)) to enable access to a cloud computing framework or other networks to provide remote data access/storage/processing, and through cooperation with an embedded operating system, access to calendars, location services, reminders, etc. The application processor 40 generally comprises a processor core (Advanced RISC Machine or ARM), and further comprises or may be coupled to multimedia modules (for decoding/encoding pictures, video, and/or audio), a graphics processing unit (GPU), communications interface (COMM) 54, and device interfaces. In one embodiment, the communication interfaces 54 may include wireless interfaces, including a Bluetooth (BT) (and/or Zigbee in some embodiments) module, 3G, 4G, or 5G module, streaming module, etc. that enable wireless communications with one or more devices of the environment 10 (FIG. 1), including the wearable device 12, and/or a Wi-Fi module for interfacing with a local 802.11 network, according to corresponding software in the communications module 52. The application processor 40 further comprises, or is coupled to, a global navigation satellite systems (GNSS) transceiver or receiver (GNSS) 56 for enabling access to a satellite network to, for instance, provide coordinate location services. In some embodiments, the GNSS receiver 56, in association with GNSS functionality in the application software 30B (e.g., as part of position determining software or integrated in the communications module 52), collects contextual data (time and location data, including location coordinates and altitude), which may be used for storage with measured data. In some embodiments, other and/or additional location technology may be used, including location through triangulation techniques.

In one embodiment, the smartphone comprises sensors 58, which may include one or any combination of physiological, contextual, and/or environmental sensors. For example, the sensors 58 may include motion (e.g., acceleration) sensing functionality (e.g., an accelerometer, inertial sensors, including a gyroscope) and/or physiological sensing functionality. In one embodiment, the sensors 58 comprise heart and/or breathing rate monitoring functionality. For instance, the sensors 58 may comprise a Philips Vital Signs Camera, or devices from other manufacturers with similar sensing functionality, to remotely measure heart and breathing rates using a standard, infrared (IR) based camera by sensing changes in skin color and body movement (e.g., chest movement), among others. The sensors 58 may also include other types of sensors, including electromyograph (EMG) sensors, impedance sensors, skin temperature sensors, environmental sensors, etc.

The device interfaces coupled to the application processor 40 may include the user interface 50, including a display screen. The display screen, which may be similar to a display screen of the wearable device user interface, may be embodied in one of several available technologies, including LCD or Liquid Crystal Display (or variants thereof, such as Thin Film Transistor (TFT) LCD, In Plane Switching (IPS) LCD)), light-emitting diode (LED)-based technology, such as organic LED (OLED), Active-Matrix OLED (AMOLED), or retina or haptic-based technology. For instance, the application software 30B may cause the rendering on the UI 50 of web pages, dashboards, questionnaires, and/or feedback (e.g., messages). Other and/or additional user interfaces 50 may include a keypad, microphone, speaker (e.g., to audibly present messages), ear piece connector, I/O interfaces (e.g., USB (Universal Serial Bus)), SD/MMC card, lighting (e.g., to provide a visualized feedback, including via different colored LEDs or different illumination patterns of the LEDs), or a tactile device (e.g., vibration motor to provide tactile feedback), among other peripherals.

Also included is a power management device 60 that controls and manages operations of a power source (e.g., battery) 62. The components described above and/or depicted in FIG. 3 share data over one or more busses, and in the depicted example, via data bus 64. It should be appreciated by one having ordinary skill in the art, in the context of the present disclosure, that variations to the above example description of the smartphone 14 may be deployed in some embodiments to achieve similar functionality.

In the depicted embodiment, the application processor 40 runs the application software 30B, which in one embodiment, includes a plurality of software modules (e.g., executable code/instructions) to carry out all or a portion of the functionality of a motivation estimation system. Further description of the application software 30B (and 30A, FIG. 2) is described in association with FIG. 5.

The communications module 52 comprises executable code (instructions) to enable the communications interface 54 and/or the radio 48 to communicate with other devices of the environment, including the wearable device 12 and/or one or more devices of the computing system 20 and/or other devices. Communications may be achieved according to one or more communications technologies, including 3G, 4G, 5G, GSM, LTE, CDMA, WCDMA, Wi-Fi, 802.11, Bluetooth, NFC, streaming technology, etc.). The communications module 52 may also include browser software in some embodiments to enable Internet connectivity. The communications module 52 may also be used to access certain services, such as mapping/place location services, which may be used to determine a context for the sensor data.

Having described the underlying hardware and software of the wearable device 12 and the electronics device 14, attention is now directed to FIG. 4, which illustrates an example computing device 66 of the computing system 20, which may be used to implement all or at least a portion of the functionality of a motivation estimation system. The computing device 66 may be embodied as an application server, computer, among other computing devices, and is also generally referred to herein as an apparatus. One having ordinary skill in the art should appreciate in the context of the present disclosure that the example computing device 66 is merely illustrative of one embodiment, and that some embodiments of computing devices may comprise fewer or additional components, and/or some of the functionality associated with the various components depicted in FIG. 4 may be combined, or further distributed among additional modules or computing devices, in some embodiments. The computing device 66 is depicted in this example as a computer system, such as one providing a function of an application server. It should be appreciated that certain well-known components of computer systems are omitted here to avoid obfuscating relevant features of the computing device 66. In one embodiment, the computing device 66 comprises a processing circuit 68 comprising hardware and software components. In some embodiments, the processing circuit 68 may comprise additional components or fewer components. For instance, memory may be separate. The processing circuit 68 comprises one or more processors, such as processor 70 (P), input/output (I/O) interface(s) 72 (I/O), and memory 74 (MEM), all coupled to one or more data busses, such as data bus 76 (DBUS). The memory 74 may include one or any combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.).

The memory 74 may store a native operating system (OS), one or more native applications, emulation systems, or emulated applications for any of a variety of operating systems and/or emulated hardware platforms, emulated operating systems, etc. In some embodiments, the processing circuit 68 may include, or be coupled to, one or more separate storage devices. For instance, in the depicted embodiment, the processing circuit 68 is coupled via the I/O interfaces 72 to template data structures (TDS) 78 and message data structures (MDS) 80, and further to data structures (DS) 82. Note that in some embodiments, one or more of these data structures 78, 80, 82, or similar with a reduced data set, may be stored at the devices 12 and/or 14. In some embodiments, the template data structures 78, message data structures 80, and/or data structures 82 may be coupled to the processing circuit 68 via the data bus 76 or coupled to the processing circuit 68 via the I/O interfaces 72 as network-connected storage devices. The data structures 78, 80, and/or 82 may be stored in persistent memory (e.g., optical, magnetic, and/or semiconductor memory and associated drives). In some embodiments, the data structures 78, 80, and/or 82 may be stored in memory 74.

The template data structures 78 are configured to store one or more templates that are used in a message definition stage to generate the messages conveying information to the user. A message for different objectives may use different templates. For example, education related messages may apply templates with referral links to educational resources, feedback on performance may apply templates with rating/ranking comments, etc. The template data structures 78 may be maintained by an administrator operating the computing system 20 and/or computing device 66. The template data structures 78 may be updated based on the usage of each template, the feedback on each generated message, among other metrics. The templates that are more often used and/or receive more positive feedbacks from the users may be highly recommended to generate the messages in the future. In some embodiments, the templates may be general templates that can be used to generate all types of messages. In some embodiments, the templates may be classified into categories, each category pertaining to a parameter. For example, templates for generating messages pertaining to heart rate may be partially different from templates for generating messages pertaining to sleep quality. The templates may vary based on motivational level. The message data structures 80 are configured to store the messages that are constructed based on the templates. The data structures 82 are configured to store user profile data including the real-time measurements of parameters for a large population of users, personal information of the large population of users, user-entered input, etc. In some embodiments, the data structures 82 are configured to store health-related information of the user and/or contextual data. The data structures 78-82 may be backend databases of the computing system 20 and/or the computing device 66. In some embodiments, however, the data structures 78-82 may be in the form of network storage and/or cloud storage directly connected to the network 18 (FIG. 1). In some embodiments, the data structures data structures 78-82 may serve as backend storage of the computing system 20 as well as network storage and/or cloud storage. The data structures 78-82 are updated periodically, aperiodically, and/or in response to a request from the wearable device 12, the electronics device 14, and/or the operations of the computing system 20 and/or computing device 66. Note that in some embodiments, the data structures 78-82 may be combined into fewer data structures or extended to additional data structures.

In the embodiment depicted in FIG. 4, the memory 74 comprises an operating system (OS) and the application software (ASW) 30C, which is described further below in association with FIG. 5. In some embodiments, the operating system may be omitted. The memory 74 further comprises a communications module (COMM) 84 that, in cooperation with the application software 30C, formats the messages to be delivered according to one or any combination of human-perceivable formats (e.g., visually, audibly, using tactile feedback, including Braille, etc.). In one embodiment, the communications module 84, in cooperation with the application software 30C, may comprise card presentation functionality. In some embodiments, card functionality resides in the application software 30C. As used herein, content cards generated for a specific parameter or plural parameters define a family of messages associated with the respective or collective parameters. For example, the content cards generated for sleep quality define a family of messages related to sleep quality, while the content cards generated for running define a family of messages related to running. The content cards may be configured to present one message per card, though in some embodiments, additional messages may be presented. Different families may define a different numbers of messages for presentation. In some embodiments, the content cards may be configured to present respective messages related to the feedback of an activity performance and/or motivation level. In some embodiments, the content cards may be configured to present messages comprising educational information, or medication information, etc. In some embodiments, the content cards may be configured to present respective messages comprising insightful analysis of the user's health-related conditions. In some embodiments, the content cards may comprise only text statements. In some embodiments, the content cards may comprise content in multiple formats including but not limited to text, audio, video, flash, hyperlink to other sources, etc. It should be appreciated that the content cards may be generated for purposes other than the examples described above, and the format of the content cards may be adjustable for presentation on different user devices. The examples set forth above are for illustrative purposes, and the present disclosure is not intended to be limiting. For instance, presentation of the messages is not limited to content card formats.

In one embodiment, the communications module 84, in cooperation with the application software 30C, is configured to receive the messages, and prepare the presentation of the content cards based on motivational level (e.g., learned, as determined from responses to questionnaires, etc.), settings pre-defined by the user and/or the configuration of each individual user device. The settings, as pre-defined or determined based on motivational level, may comprise how the user is to be notified with the content cards, for example, in a text format, in a chart format, in an audio format with low-tone female voice, in a video/flash format, and/or the combinations thereof. The settings may further comprise when and how often the user is notified with the content cards, for example, every evening around 9:00 pm, every afternoon after exercise, every week, every month, in real-time, and/or the combination thereof. In some embodiments, content card delivery and/or notification may be based on behavior and/or data. For instance, content cards may notify a user when a physiological parameter exceeds a computed or preset threshold (e.g., heart rate exceeds a computed or set threshold, when EEG signals show that the user is in a defined state(s) (e.g., alpha state), etc.). The settings may further comprise a preferred user device to receive the content card if the user has multiple devices. The configuration of each individual user device may include the size and resolution of the display screen of a user device, the caching space of the user device, etc. In some embodiments, the communications module 84, in cooperation with the application software 30C, may determine the connection status of the user device before sending the content cards. If the user device is determined to be unavailable due to power off, offline, damaged, etc., the communications module 84 may store the generated content card in memory 74 and/or upload the generated content card to the data structures 82. Once the user is detected as logged-in using one of his or her user devices, the generated content card is transmitted to the user device for presentation. In some embodiments, if the preferred user device is unavailable, the communications module 84 adjusts the content card for presentation in the logged-in user device. In some embodiments, when to present the content cards may be learned (e.g., using machine learning), such as based on feedback as to positive (or negative) efficacy and/or engagement.

The communications module 84 further enables communications among network-connected devices and provides web and/or cloud services, among other software such as via one or more APIs. For instance, the communications module 84, in cooperation with the application software 30C, may receive (via I/O interfaces 72) input data (e.g., a content feed) from the wearable device 12 and/or the electronics device 14 that includes sensed data and a context for the sensed data, data from third-party databases (e.g., medical data base, weather data, mapping data), data from social media, data from questionnaires, data from external devices (e.g., weight scales, environmental sensors, etc.), among other data. The content feed may be continual, intermittent, and/or scheduled. The communications module 84 operates in conjunction with the I/O interfaces 72 and the application software 30C to provide the messages to the wearable device 12 and/or the electronics device 14.

Execution of the application software 30C may be implemented by the processor 70 under the management and/or control of the operating system. The processor 70 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing device 66.

The I/O interfaces 72 comprise hardware and/or software to provide one or more interfaces to the Internet 18, as well as to other devices such as a user interface (UI) (e.g., keyboard, mouse, microphone, display screen, etc.) and/or the data structures 78-82. The user interfaces may include a keyboard, mouse, microphone, immersive head set, display screen, etc., which enable input and/or output by an administrator or other user. The I/O interfaces 72 may comprise any number of interfaces for the input and output of signals (e.g., analog or digital data) for conveyance of information (e.g., data) over various networks and according to various protocols and/or standards. The user interface (UI) is configured to provide an interface between an administrator or content author and the computing device 66. The administrator may input a request via the user interface, for instance, to manage the template data structure 78. Upon receiving the request, the processor 70 instructs a template building component to process the request and provide information to enable the administrator to create, modify, and/or delete the templates.

When certain embodiments of the computing device 66 are implemented at least in part with software (including firmware), as depicted in FIG. 4, it should be noted that the software can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing device 66 are implemented at least in part with hardware, such functionality may be implemented with any or a combination of the following technologies, which are known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

Note that in some embodiments of a motivation estimation system, a health engagement or coaching program may access data from a personal health record. For instance, a personal health record may be associated with a user, and is used to store health data of the user. An example of a personal health record is Apple HealthKit™. The health data comprises healthcare data (e.g., an Ultrasound report, an MRI scan, EEG data, diagnosis information, treatment information, prescriptions, etc.), personal health data (e.g., daily steps, hours of sleep, weight, BMI, etc.), and/or other environmental or contextual data associated with the user. At least a portion or all of the personal health record may be distributed across or stored on the wearable device 12 (FIG. 2), the electronics device 14 (FIG. 3), the computing device 66, or another computing resource in the environment 10. The personal health record enables the user to selectively share and manage his health data. In some embodiments, the application software 30 (e.g., 30B, 30C) receives the user's health data from the personal health record of the user, and the user is provided with an opportunity to control whether application software (e.g., 30B or 30C) can access any of the data stored in the user's personal health record.

Before describing the application software 30, a discussion on at least one theory underlying an embodiment of the motivation estimation system and hence the corresponding functionality of the application software 30 is described below, as well as a discussion on distributions (e.g., properties) that will be used to analyze the data to enable a determination of motivation estimation. Beginning with distributions and distribution analysis, distributions are typically estimated and described in terms of parameters, or features, such as mean, median, mode, variance, skewness, kurtosis, quantiles, and entropy. Specific probability density functions may be fit to the data if mathematical formulation of the distribution is desired. In certain embodiments of a motivation estimation system, skewness is emphasized as an illustrative example feature of data distribution, though it should be appreciated by one having ordinary skill in the art that other features of distribution analysis may be used. In other words, recognizing that skewness is one measure of data dispersion or variation in data distributions, in some embodiments, other measures of dispersion (measures of variation) in distributions may be used and hence are contemplated to be within the scope of the invention. In particular, skewness is one feature that refers to a measure of the asymmetry of the probability distribution of a real-valued random variable about its mean. Note that a relation between mean and median may be different depending on use of a modern non-parametric definition of skewness compared to traditional non-parametric definitions of skewness. For instance, left-skewedness does not guarantee that the mean is smaller than median, and right skewedness does not guarantee that the mean is greater than the median.

In general, distributions as used herein for certain embodiments of a motivation estimation system comprise one or more of the following features. The distribution should be a unimodal probability distribution, which is a distribution that has a single mode. Examples of unimodal distributions include normal distribution, Cauchy distribution, student's t-distribution, chi-squared distribution and exponential distribution. Considering that most data collected from living subjects falls into one of these distributions, use of a unimodal probability distribution covers the majority of applications. Another feature of distributions used in certain embodiments of a motivation estimation system is that the relation between mode, median, and mean should be either (mode median mean) or (mean median mode). In other words the median should lie between mode and mean. For most of the unimodal distributions, this is already the case. Accordingly, the distributions described herein for certain embodiments and for which (mode median mean) are referred to as positively skewed unimodal distributions, and distributions for which (mean median mode) are referred to as negatively skewed unimodal distributions. The aforementioned features ensure that the relation between mean and median can be interpreted consistently.

For purposes of illustration and simplicity in description, use herein of skewness refers to the old, non-parametric definition of skewness (versus the modern definition of skewness), where the median lies between the mean and mode. By using the non-parametric skew, thresholds may be easily defined, as well as realizing other desirable properties. For instance, a non-parametric skew is zero for any symmetric distribution (e.g., it is unaffected by a scale shift), and it reveals either left- or right-skewness equally well. Moreover, a non-parametric skew lies between −1 and +1 for any distribution. The nonparametric skew is defined as S=(mean-median)/standard_deviation. Note that other methods for determining skew may also be used in some embodiments. For symmetric distributions, skewness is zero (S=0). Further, skewness is positive for right skewed distributions, and negative for left skewed distributions.

Referring further to thresholds, absolute values greater than or equal to 0.2 (i.e. |S|>=0.2) indicate marked skewness. In certain embodiments of a motivation estimation system, 0.2 is a value used for one of the thresholds. If |S|<0.2, then the distribution is considered close to symmetric and treated as if a symmetric distribution. A sharp change in the shape of the distribution happens when |S|>=0.7746. The value, 0.7746, is used as a second threshold. Thus, |S|<0.7746, and if that condition is not met, the data is not processed as such a condition may be due to an unexpected external reason that data has become highly skewed. Note that these various threshold values may be replaced with other values in some embodiments, or omitted in part or in whole in some embodiments. In some embodiments, threshold values may be determined based on collected data (e.g., personalized). For instance, different thresholds may be suitable for different distributions of data.

Continuing with some foundational aspects to certain embodiments of a motivation estimation system, Table 1 below helps to illustrate and summarize one theory that serves at least in part as an underlying basis for motivation estimation processing, referred to as a self-determination theory:

TABLE 1

| AMOTIVATION | | EXTRINSIC MOTIVATION | | INTRINSIC MOTIVATION | |
|---|---|---|---|---|---|
| Non- | External | Introjected | Identified | Integrated | Intrinsic |
| regulation | Regulation | Regulation | Regulation | Regulation | Regulation |
| Impersonal | External | Somewhat | Somewhat | Internal | Internal |
| | | External | Internal | | |
| nonintentional | compliance, | Self-control, | Personal, | Congruence, | Interest, |
| nonvaluing | external | ego | importance, | awareness, | enjoyment, |
| incompetence, | rewards | involvement, | conscious, | synthesis | inherent |
| lack of control | and | internal | valuing | with self | satisfaction |
| | punishment | rewards | | | |
| | | and | | | |
| | | punishment | | | |

According to the Self-Determination Theory (see, e.g., Ryan R. M. and Deci, E. L., “Self-determination theory and the facilitation of intrinsic motivation, social development, and well-being”, American Psychologist, vol. 55, No. 1, 68-78, January 2000), one of the most widely accepted theories of human motivation, several types of motivation can be distinguished. These types of motivation vary in the level of self-determination with which they drive a person to engage in behavior. Motivations associated with more self-determination are more powerful in changing and maintaining behavior. The various types of motivation can be placed on a continuum (see TABLE 1 above). On one end of the continuum is amotivation, which corresponds to a state with an absolute absence of drive to perform the behavior. On the other end of the continuum is intrinsic motivation, which corresponds to a drive to engage in behavior for the inherent satisfaction and enjoyment that are caused by that behavior. Between these far ends of the continuum exists various forms of extrinsic motivation. When a behavior is performed with an extrinsic motivation, it is performed to reach certain expected outcomes of the behavior (e.g., exercising regularly in order to lose weight). Although all extrinsic types of motivation are similar in this respect, different types can be distinguished, varying in the degree to which the outcomes represent personal values. The extrinsic types towards the amotivation side of the continuum are associated with low self-determination levels. Behaviors driven by such motivations are performed for reasons that are experienced as controlled by others (e.g., I need to lose weight, otherwise my friends will not like me). In contrast, the extrinsic types towards the intrinsic motivation side are associated with higher self-determination levels. Behaviors driven by such motivations are performed for personally held values (e.g., I want to lose weight, because I think it's important to take good care of my body). Note that reference herein to self-determination behavior refers to behavior corresponding to the underlying self-determination motivation along all or a portion of the continuum illustrated in TABLE 1.

Certain embodiments of a motivation estimation system are described below with an emphasis placed on the detection of three of the types of motivation shown in TABLE 1 above, namely, amotivation, extrinsic motivation, and intrinsic motivation. In the description that follows, it is assumed that the user is following a coaching and engagement program aimed at improving certain (measurable and quantifiable) behavior, that the user's baseline level of behavior (e.g., step count) is measured, and that a (numeric) target is set (e.g., 10,000 steps per day). The target generally (though not necessarily) represents an improvement in behavior compared to the baseline period. The target may be a higher value (e.g., step count, active minutes, hours of sleep) or a lower value (e.g. calorie intake, minutes spent on social media) than the baseline value, depending on the type of target.

Attention is now directed to FIG. 5, which illustrates an example software architecture for the application software (ASW) 30 (e.g., 30A, 30B, 30C), which is used to implement functionality of certain embodiments of a motivation estimation system. The application software 30 may reside entirely within a single device, including the wearable device 12 (FIG. 2), the electronics device 14 (FIG. 3), or the computing device 66 (FIG. 4), or in some embodiments, the functionality of the application software 30 may be distributed among plural devices (e.g., the wearable device 12, the electronics device 14, and/or the computing device 66). In one embodiment, the application software 30 comprises executable code/instructions comprising an optional (as indicated by the dashed box) normalization module (NRMLZ) 86, a distribution parameter calculation module (DSTRBCLC) 88, a motivation estimation module (MVEST) 90, an intervention adaptation module (INTADPT) 92, and a target calculation module (TGTCLC) 94. It should be appreciated by one having ordinary skill in the art, in the context of the present disclosure, that variations to the architecture may be implemented, including fewer and/or additional modules to achieve the same and/or additional functionality, and hence such variations are contemplated to be within the scope of the disclosure. In some embodiments, functionality corresponding to one or more of the modules 86-94 may be implemented in hardware. For illustrative purposes, and in the interest of brevity, the application software 30 is primarily described below as being executed by the computing device 66 (with interventions or engagement with the user, including message presentation, achieved at the wearable device 12 and/or the electronics device 14), with the understanding that processing may be achieved in other devices or among plural devices in some embodiments.

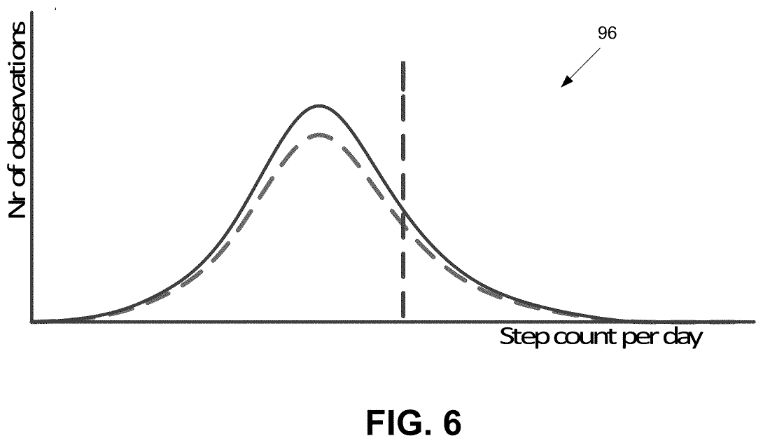

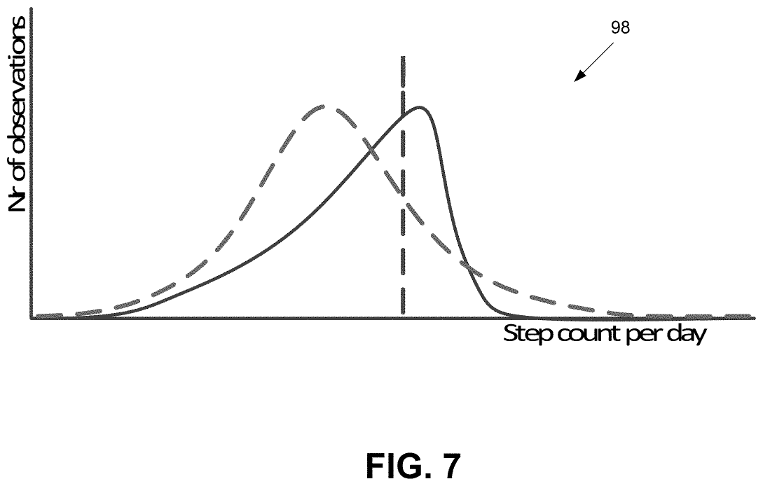

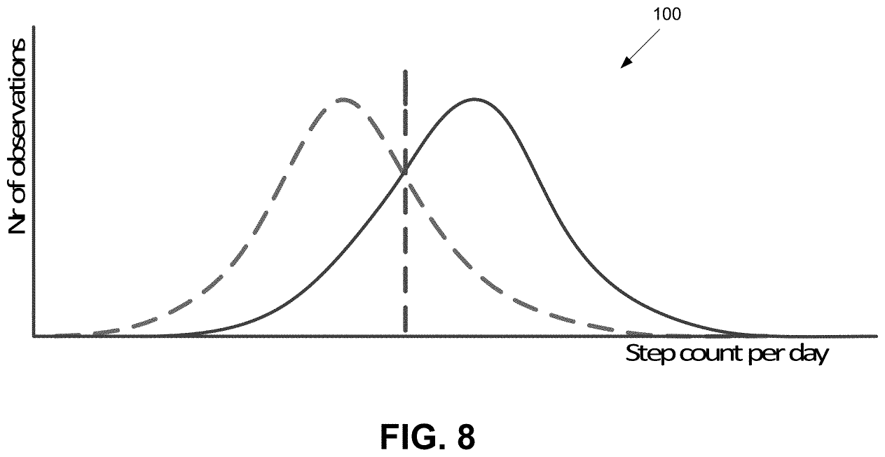

In general, the application software 30 may run, or operate in conjunction with, a health engagement application, including a coaching and engagement application, as explained above. Note that reference to a coaching and engagement application for a health engagement application is illustrative of one example implementation, and that in some embodiments, any type of user-engagement and/or intervention application in the fields or endeavors of health, finance, business, medicine, therapy, etc. may be used where motivation estimation is beneficial in influencing changes in behavior. The coaching and engagement application (or application software 30) implements, or causes the implementation of, an intervention/engagement, including a coaching message and/or other intervention/engagement, based on input gathered from the wearable device 12 and/or electronics device 14, including physical activity, behavior, user state, and context, the coaching message intended to influence the user in, for instance, advancing progress towards a goal (e.g., entered by the user, including losing weight, building endurance, etc.). Before describing the modules 86-94 of the application software 30, an example of motivation level estimation functionality of the application software 30 using distribution analysis is illustrated in association with FIGS. 6-8 in the case of walking behavior. Referring to FIG. 6, shown is an example distribution 96 reflecting amotivation, with a dashed curve indicating baseline behavior (step count per day, as shown on the x-axis, with number of observations on the y-axis)) and a solid curve indicating observed behavior relative to a target (indicated by the dashed vertical line). As shown, if people are amotivated to become more active and reach the step target, their behavior is independent of the target and the step data is distributed normally, with the mean comparable to baseline level. Notably, the normal distribution has a peak that is lower than the target. Referring to FIG. 7, shown is an example distribution 98 reflecting an extrinsically motivated person, again with a dashed curve indicating baseline behavior (step count per day, as shown on the x-axis, with number of observations on the y-axis)) and a solid curve indicating observed behavior relative to a target (indicated by the dashed vertical line). As illustrated in FIG. 7, if people are extrinsically motivated by the target, they actively try to reach the target, but are not motivated to take more steps after reaching the target. This behavior results in a skewed distribution, with a peak just above the target. FIG. 8 shows an example distribution 100 reflecting an intrinsically motivated person, with a dashed curve indicating baseline behavior (step count per day, as shown on the x-axis, with number of observations on the y-axis)) and a solid curve indicating observed behavior relative to a target (indicated by the dashed vertical line). As illustrated in FIG. 8, if people are intrinsically motivated to walk, their behavior is independent of the target, but in general they are likely to reach the target. Thus, the step data is distributed normally, with a mean higher than the target.

Referring again to FIG. 5, one embodiment of the application software 30 performs baseline measurements/data collection (distribution of data in the absence of a target) for a user, target measurements/data collection (distribution of data in the in the presence of a set target) for the user, target distribution analysis with respect to a set target, and estimation of a motivational state (e.g., amotivated, extrinsically motivated, intrinsically motivated) of the user. Note that reference herein to measurement/data collection refers to the circumstances where measurements may take place at sensors (e.g., located at the wearable device 12 and/or the electronics device 14, FIG. 1), and data collection may likewise take place at the same devices and/or at a device remote from where measurements are taken, including at the remote computing system 20 where data collection is performed based on receipt of a communication of measurements from the wearable device 12 and/or electronics device 14. It is noted that data collection also includes data that is received based on indirect measurements or non-measured information, including where data records or other information are obtained based on data inputted by personnel as observed from measurements and/or data entered by a user or other personnel (e.g., demographic data) and/or other devices. In particular, and referring to the functionality of the distribution parameter calculation module 88, the distribution parameter calculation module 88 provides for baseline measurements. A user is monitored (e.g., via the wearable device 12, electronics device 14, etc.) for a determined time, ideally until representative data is collected by the distribution parameter calculation module 88 in sufficient amount that enables estimation of a probability density function. During these baseline measurements, there is no target presented to the user. In one embodiment, the distribution parameter calculation module 88 fits the baseline data to a normal distribution, so the baseline collection is continued until ISI (the absolute value of the skewness measure) becomes closer to zero. In some embodiments, baseline distribution is also skewed, which is described below. For real life data, it is a valid assumption that data will get closer to normal distribution.

Note that in some embodiments, baseline measurements may not be performed to determine a motivational level, such as when the target is known to be selected in a reasonable manner (e.g., from representative, similar population data, or from a thorough and accurate assessment via a questionnaire as described further below), though implementations without baseline data may heighten the risk that the target may be set unreasonably high or low.

The distribution parameter calculation module 88 also provides for target measurements. Target measurements are defined as measurements performed for the periods when a user is presented with a target. The target, or similarly target behavior, is ideally set based on the baseline measurements, and it is set to represent an improved behavior relative to the baseline. In other words, the application software 30 transitions, after a suitable amount of baseline data has been collected, from baseline measurements to target measurements that are based on the baseline measurements. In one embodiment, the distribution parameter calculation module 88 may determine the target to be equal to the median of the baseline plus delta (Δ). For example, the delta can be a pre-set value (e.g., 1000 steps). In some embodiments, the delta may be calculated from the user's data (e.g., Δ=standard deviation (baseline data)). In some embodiments, the target may be set according to other methods, including more generally as a function of the baseline data (e.g., transforming features of the baseline data). Similar to the baseline period, the user is monitored (e.g., via the wearable device 12, electronics device 14, etc.) for a period of time until a reliable probability density distribution may be estimated.

The distribution parameter calculation module 88 further provides for target distribution analysis with respect to the set target. Non-parametric skew statistics for both baseline and target distributions are calculated. The following table (TABLE 2) is used by the motivation estimation module 90 in conjunction with the calculations performed by the distribution parameter calculation module 88 to estimate the motivation level/type. As a brief introduction to Tables 2 and 3, the first column refers to a target distribution, which corresponds to the distribution of data collected when target behavior is present. The target distribution may be categorized according to a determined skewness (e.g., close to symmetric, left skewed (negative), or right skewed (positive)) based in part on threshold values, as described further below. In this example embodiment, TABLE 2 and TABLE 3 apply when |Sbaseline|<0.2 and 0.2≤|Starget|<0.7746. Note that the choice of threshold values (e.g., 0.2 and 0.7746) are illustrative of one example, and in some embodiments, other threshold values may be used. For instance, the threshold values may be determined from collected data, enabling a more personalized and/or dynamic (adaptive) determination of skewness. Continuing with an explanation of the tables, ATarget is a feature that is calculated from the data collected when target behavior is present. Target is the target behavior that is presented to the user. In one embodiment, the Target is extracted from the baseline data. In some embodiments, the Target may be estimated without baseline data. For instance, as explained above, where baseline data is unavailable, the Target may be determined from population data, responses to questionnaires, etc. For the examples of Tables 2 and 3, the Target is the mode of the baseline data, and ATarget is the mode of the data when the target is present. In TABLE 2 (and TABLE 3), close to symmetric refers to |Starget|<0.2, left skewed refers to Starget≤−0.2, and right skewed refers to Starget≥0.2. In general, if Starget>0, then it is positive or right-skewed, and if negative it is negative or left-skewed. In some embodiments, the distribution parameter calculation module 88 may also calculate the mode and median for the target distribution. If mode can be estimated reliably, mode is used, otherwise, median. In other words, in some embodiments, Atarget (and Target) may correspond to either the mode, median, or mean, where TABLE 2 and TABLE 3 reveals the motivational levels determined from analysis of distribution statistics (ATarget), or simply distribution, with respect to a target and the skewness of the distribution based on the baseline data. It should be appreciated by one having ordinary skill in the art, in the context of the present disclosure, that the motivational levels expressed in tables 2 and 3 change depending on the calculated features (e.g., baseline and target distribution features are compared differently to reach motivational levels). For instance, TABLE 2 represents a function that may be used to estimate the motivational levels/types when the mode (or in some embodiments, median or mean) of the collected data is available. If a different feature is calculated (e.g., a quantile of the baseline and target data), a different function (and hence different table) is used.

TABLE 2

| | ATarget = | Atarget > | Atarget < |
|---|---|---|---|
| Target Distribution | Target | Target | Target |
| Close to | Extrinsic | Intrinsic | Amotivation |
| symmetric | motivation | motivation | |
| Left skewed | Extrinsic | Extrinsic | Amotivation |
| (negative) | motivation | motivation | |
| Right skewed | Extrinsic | Extrinsic | Extrinsic |
| (positive) | motivation | motivation | motivation |

As shown in TABLE 2, the user motivational state (e.g., extrinsic motivation, intrinsic motivation, and amotivation) is inferred by the motivation estimation module 90 from the observed target distribution data and its relationship to the set target. In one embodiment, the skewness is determined using S=(mean-median)/standard_deviation, where mean, median, and standard deviation are calculated from the target data distribution. In some embodiments, skewness may be estimated for baseline using population data, responses to questionnaires, among other mechanisms as similarly described for estimating the target in the absence of baseline data. As one example approach, a user may be asked to report periodically (e.g., every hour) every time the user walks and associated information (e.g., duration, time started, pace, etc.), and based on the data, the user step data may be estimated and a baseline distribution for the estimated step data may be obtained. As explained above, the TABLES 2 and 3 use skewness as one measure of change (variation) in the distribution of the data to estimate motivation, but it should be appreciated by one having ordinary skill in the art that other measures of variation may be used in some embodiments and are hence contemplated to be within the scope of the invention.

In some embodiments, the application software 30 (e.g., motivation estimation module 90 in conjunction with the distribution parameter calculation module 88) may populate TABLE 2 with more detailed motivation information, as shown in TABLE 3 to also reflect the degree (e.g., sub-levels, including the various regulatory styles of extrinsic motivation shown in TABLE 1) of the motivation.

TABLE 3

| | ATarget = | Atarget > | Atarget < |
|---|---|---|---|
| Target Distribution | Target | Target | Target |
| Close to symmetric | M4-M5 | M5 | M0 |
| Left skewed | M1-M2 | M2-M3 | M0-M1 |
| (negative) | | | |
| Right skewed | M3-M4 | M4-M5 | M1-M3 |
| (positive) | | | |

In TABLE 3, which is drawn on the degrees of motivation from TABLE 1, M0=amotivation, non-regulation, M1=extrinsic motivation, external regulation, M2=extrinsic motivation, introjected regulation, M3=extrinsic motivation, identified regulation, M4=extrinsic motivation, integrated regulation, and M5=intrinsic motivation, intrinsic regulation. Note that the determinations in TABLE 2 (e.g., extrinsic, intrinsic, etc.) are coarser (e.g., rounded) than the deterinations (e.g., M4-M5, M1-M2, etc.) of TABLE 3. Aside from this fact, the cells in both tables are about the same. One difference is that in TABLE 3, there is a more fine-grained motivation estimation performed by the logic determining the connections between how symmetric or skewed the distribution is and the comparisons of the ATarget and Target (similar to TABLE 2). In other words, by using the same information and the same or a similar analysis, different levels of detail about the motivation can be provided. In one sense, the difference in tables may be thought of a difference in quantization of motivation levels. Thus, an accumulation of a large amount of data for more fine-grained quantifications of the motivation levels may be achieved as reflected in TABLE 3.

Note that in some embodiments (e.g., due to a specific schedule), the user baseline data may be skewed, and there may not be enough time for the distribution parameter calculation module 88 to collect more data until it becomes normally distributed. In such embodiments, changes in the data distribution may be also compared among each other, which may provide additional insights of how the motivation level is fluctuating over time. To handle these scenarios, the motivation estimation module 90 in conjunction with the distribution parameter calculation module 88 compares the target skewness with baseline skewness to decide if the data has skewed more or less, as shown in TABLE 4 below.

TABLE 4

| | ATarget = | Atarget > | Atarget < |
|---|---|---|---|
| Target Distribution | Target | Target | Target |
| \|Starget − | M4-M5 | M5 | M0 |
| Sbaseline\| ≤ 0.1 | | | |
| Starget − | M1-M2 | M2-M3 | M0-M1 |
| Sbaseline < −0.1 | | | |
| Starget − | M3-M4 | M4-M5 | M1-M3 |
| Sbaseline > 0.1 | | | |

In some embodiments, an interaction (e.g., the distance) between the set target and mode may be used as an additional parameter. Note that an interaction, as is known, refers to a statistical analysis, where the relation or interaction between independent variables with each other may be analyzed with respect to dependent variables. Other independent variables (e.g., aside from distance) may be included in the analysis, including mean, median, amount of data, type of data, context of data (e.g., time of data, place of data, etc.), among others. The relation or interaction between these different variables may be calculated and analyzed for various groups of variables, including variable pairs, triplets, etc. That is, the application software 30 may compute the distance (or other interaction) between the target and Atarget (e.g., mode or median) of the target distribution. The distance may be used as an additional parameter to determine the confidence of the motivation level estimation performed by the motivation estimation module 90. If the mode is close to the target and the distribution is skewed as described above, the level of extrinsic motivation due to the target may be estimated to be higher. In contrast if the distance between the target and the mode is above a threshold, this can be used as an indicator that the user is not motivated by the target (e.g., that he is either amotivated or intrinsically motivated). For example, in a case where the distance between the target and Atarget is larger than a threshold (e.g., for step data, the threshold may be computed as equal to the mean(baseline)/2 steps), then even if the distribution is right skewed, then it is likely the user is more intrinsically motivated (M5) than extrinsically motivated. It should be appreciated by one having ordinary skill in the art, in the context of the present disclosure, that the example of distance explained above is but one example based on using features of mode, median, or mean, and that in some embodiments, including when other features are used (e.g., quantiles), other mechanisms for determining motivation and quality may be used. Ultimately, certain embodiments may use the collected data to estimate the motivation and quality indicators.

The application software 30 further comprises the intervention adaptation module 92 and target calculation modules 94. As explained above, motivation assessment can be valuable for any application that is aiming for a behavior change in humans. One example use is depicted in FIG. 5, where it is shown that estimated motivation from motivation estimation module 90 is used to adapt the intervention parameters of the intervention adaptation module 92, which can result in a new target (TGT) being calculated by the target calculation module 94. In general, the target calculation module 94 is driven by the intervention adaptation module 92, the target calculation module 94 using the input data selected by the intervention adaptation module 94 to calculate a target in this example. The intervention adaptation module 92 may control additional functions, including the type of data collected and analyzed, types of messaging, etc., as explained further below. In this example, if the user is extrinsically motivated by the target, the target may be kept the same or increased. If the user is amotivated by the target, the target may be changed to be closer to the baseline values or even less challenging than baseline. Although depicted in FIG. 5 as the intervention adaptation module 92 triggering the target calculation module 94 to provide a target value, in some embodiments, the intervention adaptation module 92 may trigger other and/or additional adaptations as indicated above. For instance, the current intervention module (e.g., running application or program) may be terminated and a new intervention module may be instantiated (e.g., terminate and replace a get active walking program with an education program.) If the user is intrinsically motivated, the target may be dropped, or a new intervention module (requiring a different type of target, for example increasing the sleep duration, instead of increasing the step count) may be put into action. Clearly other and/or additional intervention parameters may also be adapted as a function of the estimated motivation, including intervention parameters related to message characteristics (e.g., delivery time, message content, message size), communication and coaching characteristics (e.g., styles), etc. The observed motivation may also trigger (by the intervention adaptation module 92) collecting different types of data as a result of the observed motivation. For example, location may start to be tracked to better understand why the person is amotivated, extrinsically motivated, or intrinsically motivated. In effect, the application software 30 may implement an intervention/action or trigger the implementation of an intervention/action by another system or device in an attempt to influence a change in behavior of the user.

The application software 30 also comprises a normalization module 86, which takes context parameters (CTX) into account when providing a user data (UD) distribution. This is an optional pre-processing of the user data. In certain conditions, it can be very beneficial, and even necessary, to normalize the user data to be able to reduce bias, reduce modelling specific external factors, and/or reduce modelling external events while calculating the user data distribution. Especially for people that travel, have varying schedules, work shifts, have suffered injuries, changed context, experience varying weather conditions, are experiencing a holiday, etc., normalization may take on higher importance. A simple way to perform the normalization is to (i) determine the context parameters that are expected to influence measured target behavior, (ii) group baseline and target data based on the context parameters, and (iii) compare baseline and target data that share the same context parameters (e.g., that belong to the same clusters). To combine and collectively analyze all collected data (e.g., data from all clusters), for each individual cluster, the target data is normalized using the baseline data in the same cluster. Some normalization options include (target data−mean(baseline data))/(standard deviation (baseline data)) or (target data)/(mean(baseline data)).

Alternatively, the normalization with regard to the external factors mentioned above (e.g., weather, schedule, etc.) may also be done by weighting the corresponding data by taking context parameters into account. The weighting can be considered as a transformation function, which has the context as parameters. The effect of the transformation function may be to emphasize or de-emphasize data points depending on the context parameter to ensure that all data points collected in different contexts can be collectively analyzed. An example for weather-based normalization is the following: If the weather is not suitable to achieve the target (e.g. walking), this should be taken into account so that the user is not mistakenly identified as amotivated. The reverse is also true: if the weather is unusually suitable to achieve the target (e.g., sunny days in normally harsh winter), the user should not be mistaken to be intrinsically motivated. The weight of steps taken during bad weather may be greater than steps taken during good weather. In one example transformation, one step taken in bad weather while being outdoors may be considered as 2 units, while one step taken in normal weather is 1 unit, and one step taken in good weather may be considered to be 0.5 units. The user data distribution is then determined based on the normalized unit data.

The above-described embodiments of a motivation estimation system provide for a method to unobtrusively quantify and measure a person's level/type of motivation. One advantage over existing methods is that certain embodiments of a motivation estimation system do not require the user to complete dedicated questionnaires, and provides a mechanism for unobtrusive, continuous, and ubiquitous motivation assessment. However, in some embodiments, the use of questionnaires and responses to questionnaires (questionnaire response data) may be beneficial. For instance, certain embodiments of a motivation estimation system may use the responses to questions to score messages along the self-determination continuum and hence provide an initial set of messages that are suited to (e.g., matching) the motivational level of the user. In one embodiment, and in general, motivation levels of each message presented to a user or users of a control group are scored (e.g., scored, or similarly, labeled or annotated) based on responses of the control groups to the questionnaires. Then, the motivational levels of these messages are updated based on the responses and/or behavior of the user (non-control group). In other words, the scoring (labeling, annotation) of the messages is based on the control group data.. Subsequently, the data from real users (non-control group) is collected and there is a re-computation of the scores based on the collected data from the real users. More specifically, and in one embodiment, the motivation estimation system (e.g., the application software 30, though other software may be used) collects training data (e.g., representative data for representative users) and then uses the collected data to guide the adaptation of the messages based on the self-determination continuum. In one implementation, prior to the development (or deployment) of the motivation estimation functionality, data from various subjects may be collected. Such subjects (e.g., test subjects or test users) may be part of a control group whose responses to a motivation assessment questionnaire are analyzed to enable the identification of a link between message features and motivation levels (e.g., M0 to M5 from TABLE 3). The questionnaire can be repeated at regular intervals (e.g., every week).

In one embodiment, coaching messages may be delivered to the test users, and the test users may be asked to evaluate the coaching messages to ascertain which messages are suitable for certain type of motivations. For instance, after each coaching message, feedback is requested from the users, where an example feedback may be of the form of a question asking if the current message is suitable. As a result of the responses, the motivation estimation system learns what types of messages are suitable for respective types of motivation levels. Such collected information may be represented as a table. Moreover, specific lower level features may be extracted from messages (e.g., content of card: educational, feedback; type of card: text, audio, video; time of delivery: in-situ, pre-set times, etc.) and a similar tabulation as above of features and motivational levels may be made. In effect, an embodiment of the motivation estimation system learns how the low-level features of messages are linked to the respective motivation levels. If it is determined that a certain low-level feature is strongly linked (e.g., correlated) to a particular motivation level, in any subsequent design of new cards, this low-level feature may be introduced or omitted. For example, if it is learned that for intrinsically motivated users, providing statistical analysis is desired, and giving actionable advice is not desired, cards intended for such users may be designed to include a data overview and analysis, and not include any activity advice. A relevant low-level feature (per motivation level) may be learned automatically (e.g., using machine learning) from the training data collected from the test users, where through the training data, it is learned how certain message features are linked to motivational levels. Note that during the training data collection, there should be a sufficient amount of input data (e.g. motivational questionnaires and assessment of individual messages).

Having established the control group data, implementation focuses on the real user (e.g., users that make use of a coaching program that uses the motivation estimation functionality of the application software 30). For such users, the continuous user input and feedback experienced among the control group users is not present for the real user. In one implementation, when enrolling in the program (e.g., a coaching and engagement program with the motivation estimation functionality), the real user is asked to complete a motivation assessment questionnaire. Based on the responses to that questionnaire, the motivational level of the user is determined, and suitable messages (e.g., suitable to their motivational level) may be selected based on the above-referenced tabulation(s) derived from the training data. Changes in a real users motivation level are automatically (i.e., without requiring additional user input) evaluated by the application software 30 (as described above for FIG. 5), which in the case of messages, results in the adjustment (via intervention adaptation module 92) of the message characteristics (e.g., type of message, such as educational, style of message, such as positive, negative, etc.), or more generally, an update of the scoring takes place. The questionnaire thus provides a mechanism in some embodiments of a motivation estimation system to determine an initial motivation level of a user to enable the presentation of messages more suited to their motivational level. With the collection of data, calculation of a motivation score, and the user receiving messages, enough resources are at play to train a machine learning engine that enables adaptation of the relevance of the messages to the motivation levels. The questionnaires provide a mechanism to initialize the motivation estimation functionality, and then the machine learning features of the motivation estimation system learns a link between the messages and the motivation scores.

As described above, the application software 30 provides for a distribution of collected baseline measurements, such data used to estimate a reasonable target that may be used for motivation estimation. However, in some embodiments, the questionnaire data may be sufficiently comprehensive and accurate to establish a target (or used to calculate a target) without the need for baseline measurements. For instance, if a user already knows that they do 10,000 steps on average, then a target may be set to 12,000 steps (without collecting baseline measurements) and the motivation can be estimated as discussed in the description above pertaining to the explanation of TABLE 2. In some embodiments, despite the thoroughness of the data collected from a questionnaire, baseline measurement collection is still performed given the disparities in measurements by wearable devices among different manufacturers. That is, behavioral data measured using wearable manufacturer A and a questionnaire required to be completed by manufacturer B for providing motivation estimation functionality may result in the target estimate being erroneous. Consider an example, where the user uses a wearable device made by Manufacturer A. The wearable device measures behavior of the user, such as making a determination that the user has performed an average number of steps equal to X. Subsequently, the user switches to using a wearable device from Manufacturer B, which uses a different mechanism/algorithm to determine an average number of steps (e.g., equal to Y). In one instance, there are no baseline measurements, and the user inputs (e.g., to respond to a questionnaire) that he performs an average number of steps equal to X, and the target is estimated based on X. However, since the average number of steps Y determined by the wearable device from Manufacturer B is different than that (e.g., X) determined from the wearable device of Manufacturer A, the motivation level may be erroneous. In another instance, the first baseline measurement may be performed with the wearable device from Manufacturer B, hence estimating a target from that measure. In this instance, the result is an accurate motivation estimation. It is noted that the baseline measurements are used to estimate a reasonable target, enabling the tracking of changes in the motivation of a user.

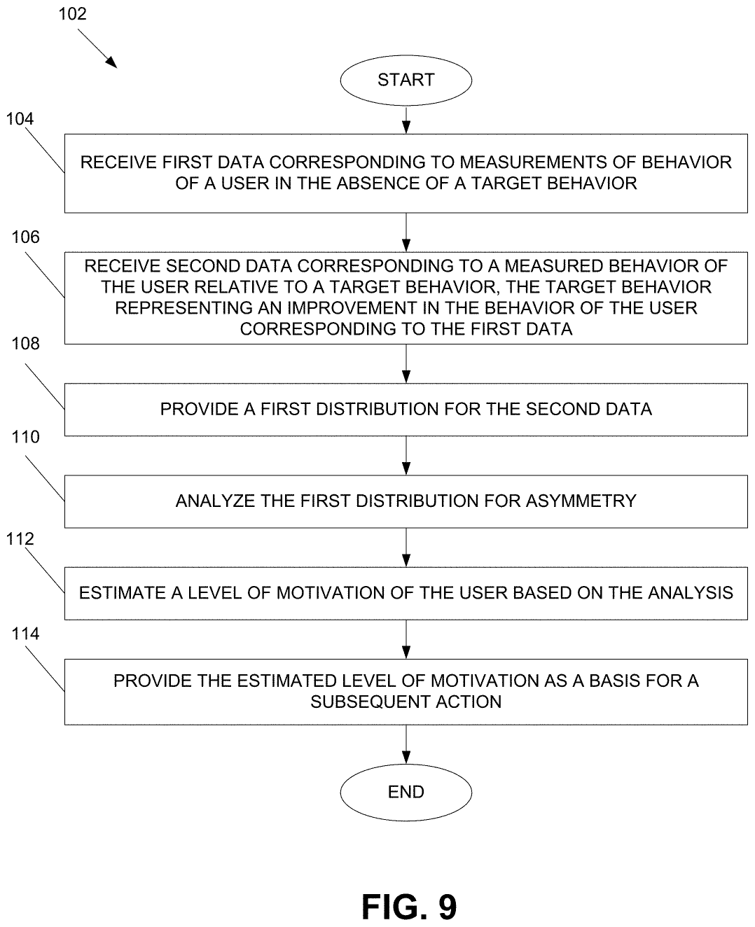

Having described certain functionality of the application software 30 illustrated in FIG. 5, it should be appreciated that one embodiment of an example motivation estimation method, depicted in FIG. 9 and denoted as method 102, which is shown bounded by a start and end, comprises receiving first data corresponding to measurements of behavior of a user in the absence of a target behavior (104); receiving second data corresponding to a measured behavior of the user relative to a target behavior, the target behavior representing an improvement in the behavior of the user corresponding to the first data (106); providing a first distribution for the second data (108); analyzing the first distribution for asymmetry (110); estimating a level of motivation of the user based on the analysis (112); and providing the estimated level of motivation as a basis for a subsequent action (114).

Any process descriptions or blocks in flow diagrams should be understood as representing modules, segments, or portions of code which include one or more executable instructions for implementing specific logical functions or steps in the process, and alternate implementations are included within the scope of the embodiments in which functions may be executed out of order from that shown or discussed, including substantially concurrently or in reverse order, depending on the functionality involved, as would be understood by those reasonably skilled in the art of the present disclosure. In some embodiments, one or more steps may be omitted, or further steps may be added.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

1. At least the following is claimed:
An apparatus, comprising:
a memory comprising instructions; and
one or more processors configured to execute the instructions to:
receive first data corresponding to measurements of behavior of a user in the absence of a target behavior;
receive second data corresponding to a measured behavior of the user relative to a target behavior, the target behavior representing an improvement in the behavior of the user corresponding to the first data;
provide a first distribution for the second data;
analyze the first distribution for asymmetry;
estimate a level of motivation of the user based on the analysis; and
provide the estimated level of motivation as a basis for a subsequent action.

2. The apparatus of claim 1, wherein the estimated level of motivation of the user corresponds to at least amotivation, extrinsic motivation, or intrinsic motivation, wherein each level varies in an amount of self-determination behavior of the user.

3. The apparatus of claim 2, wherein the estimated level of motivation further corresponds to any one of a plurality of sub-levels of the extrinsic motivation.

4. The apparatus of claim 1, wherein the one or more processors are further configured to execute the instructions to analyze the first distribution by computing features of the first distribution and using the computed features as a basis for the estimation.

5. The apparatus of claim 4, wherein the computed features correspond to a measure of variation in the first distribution.

6. The apparatus of claim 4, wherein the computed features correspond to skewness.

7. The apparatus of claim 1, wherein the one or more processors are further configured to execute the instructions to set the target behavior based on the first data, wherein the one or more processors are configured to execute the instructions to transition from receiving the first data to receiving the second data after setting the target behavior.

8. The apparatus of claim 7, wherein the one or more processors are further configured to execute the instructions to set the target behavior after a sufficient amount of the first data is collected to enable formation of a second distribution.

9. The apparatus of claim 8, wherein the one or more processors are further configured to execute the instructions to set the target behavior by using features of the second distribution.

10. The apparatus of claim 8, wherein the one or more processors are further configured to execute the instructions to estimate by computing features of the first and second distributions and using these computed features for the estimation.

11. The apparatus of claim 10, wherein the one or more processors are further configured to execute the instructions to determine an interaction between the target behavior and the features of the first distribution, wherein the interaction is used to determine a confidence of the estimation.

12. The apparatus of claim 1, wherein the one or more processors are further configured to execute the instructions to provide the estimated level of motivation by implementing, or causing the implementation of, one of:
adapting one or more intervention parameters, wherein the one or more intervention parameters comprise the target behavior, messaging characteristics, communication characteristics, or coaching characteristics; or
replacing a running intervention module with another intervention module, wherein running intervention module or the another intervention module comprises an application run by the one or more processors and intended to change behavior of the user.

13. The apparatus of claim 1, wherein the one or more processors are further configured to execute the instructions to receive contextual data and normalize the first and second data based on the contextual data.

14. The apparatus of claim 1, wherein the one or more processors are further configured to execute the instructions to provide a distribution for the second data, wherein the one or more processors are further configured to execute the instructions to analyze the distribution for asymmetry.

15. The apparatus of claim 14, wherein the one or more processors are further configured to execute the instructions to estimate the level of motivation of the user by comparing a change in the asymmetry of the first distribution with a change in the asymmetry of the distribution corresponding to the first data.

16. The apparatus of claim 1, wherein the one or more processors are further configured to execute the instructions to:
receive first questionnaire response data from a control group;
associate message types and features to motivational levels based on the receipt of the first questionnaire response data;
receive second questionnaire response data from the user;
determine a motivational level of the user based on receipt of the second questionnaire response data; and
provide messaging to the user based on comparing the motivational level of the user to one of the message types and features.

17. The apparatus of claim 16, wherein the one or more processors are further configured to execute the instructions to determine the target behavior based on receipt of the second questionnaire response data.

18. A method implemented by one or more processors executing instructions, the method comprising:
receiving first data corresponding to measurements of behavior of a user in the absence of a target behavior;
receiving second data corresponding to a measured behavior of the user relative to a target behavior, the target behavior representing an improvement in the behavior of the user corresponding to the first data;
providing a first distribution for the second data;
analyzing the first distribution for asymmetry;
estimating a level of motivation of the user based on the analysis; and
providing the estimated level of motivation as a basis for a subsequent action.

19. The method of claim 18, further comprising implementing, or causing the implementation of, one of:
adapting one or more intervention parameters, wherein the one or more intervention parameters comprise the target behavior, messaging characteristics, communication characteristics, or coaching characteristics; or
replacing a running intervention module with another intervention module, wherein running intervention module or the another intervention module comprises an application run by the one or more processors and intended to change behavior of the user.

20. A non-transitory, computer readable medium comprising instructions that, when executed by one or more processors, causes the one or more processors to:
receive first data corresponding to measurements of behavior of a user in the absence of a target behavior;
receive second data corresponding to a measured behavior of the user relative to a target behavior, the target behavior representing an improvement in the behavior of the user corresponding to the first data;
provide a first distribution for the second data;
analyze the first distribution for asymmetry;
estimate a level of motivation of the user based on the analysis; and
provide the estimated level of motivation as a basis for a subsequent action.
